SEQUENCE LISTING (9 sequences)

SEQ ID NO: 1 - DNA, 27 bp, Artificial Sequence
  Synthetic construct
gtccactaccgctatcatggcggtctc

SEQ ID NO: 2 - DNA, 26 bp, Artificial Sequence
  Synthetic construct
gagaccaggagacaggaccgaccaaa

SEQ ID NO: 3 - DNA, 861 bp, Artificial Sequence
  Synthetic construct
ggagggttgcgtttgagacgggcgacagatcatatgcgggaggcggtgatcgccgaggtgagcacgcagctgtccgaggtggtgggcgtcatcgagcgccacctggagccgacgctgctggccgtccacctgtacggcagcgccgtggacggcggcctgaagcctcactccgacatcgatctgctggtgacggtgaccgtccgcctggacgagactactcgccgcgctctgatcaacgacctgctggagacgtccgcctcccccggggagagcgagatcctccgggctgtggaggtgaccatcgtggtgcacgacgacatcatcccttggcgctaccccgctaagcgcgagctgcagttcggtgagtggcagcggaacgacatcctggcgggcatcttcgagccggctaccattgacatcgacctggctatcctgctgacgaaggcccgcgagcatagcgtggcgctggtcggccccgccgcggaggagctgttcgaccctgtgccggagcaggacctgttcgaggctctgaacgagacgctgaccctctggaactcccctcccgactgggccggtgacgagcgcaacgtggtcctgacgctgtcgcgcatctggtactcggccgtgaccggcaagatcgcgcccaaggacgtggcggcggactgggcgatggagcgcctccccgcgcaataccagcccgtgatcctggaggcccgccaggcgtacctgggccaggaggaggaccgcctggcctcccgcgcggaccagctggaggagttcgtgcactatgtgaagggcgagatcactaaggtggtgggcaagtaaggatccatcagttctggacgagcgagctgtcgtccg

SEQ ID NO: 4 - DNA, 337 bp, Artificial Sequence
  Synthetic construct
ggagggttgcgtttgagacgggcgacagatcatatgcgggaggcggtgatcgccgaggtgagcacgcagctgtccgaggtggtgggcgtcatcgagcgccacctggagccgacgctgctggccgtccacctgtacggcagcgccgtggacggcggcctgaagcctcactccgacatcgatctgctggtgacggtgaccgtccgcctggacgagactactcgccgcgctctgatcaacgacctgctggagacgtccgcctcccccggggagagcgagatcctccgggctgtggaggtgaccatcgtggtgcacgacgacatcatcccttggcgctacc

SEQ ID NO: 5 - DNA, 35 bp, Artificial Sequence
  Synthetic construct
gcgatgaccgctatcatggccgctaccgttgatag

SEQ ID NO: 6 - DNA, 31 bp, Artificial Sequence
  Synthetic construct
atggatcagttctgggacaggaccgcatcgc

SEQ ID NO: 7 - DNA, 45 bp, Artificial Sequence
  Synthetic construct
gtcactaccgctatcatggcggtctctggccgctaccgttgatag

SEQ ID NO: 8 - DNA, 41 bp, Artificial Sequence
  Synthetic constuct
atggatcagttctgggagaccaggagacaggaccgaccaaa

SEQ ID NO: 9 - DNA, 11 bp, Artificial Sequence
  Synthetic construct
ccagagnnnnn

(19) United States
(12) Patent Application Publication
(10) Pub. No.: US 2015/0203839 A1
(43) Pub. Date: Jul. 23, 2015

(54) Compositions and Methods for High Fidelity Assembly of Nucleic Acids

(71) Applicants: Joseph Jacobson, Newton, MA (US); Daniel Schindler, Newton, MA (US); Scott S. Lawton, Bedford, MA (US)

(72) Inventors: Joseph Jacobson, Newton, MA (US); Daniel Schindler, Newton, MA (US); Scott S. Lawton, Bedford, MA (US)

(73) Assignee: Gen9, Inc., Cambridge, MA (US)

(21) Appl. No.: 14/241,242

(22) PCT Filed: Aug. 23, 2012
(86) PCT No.: PCT/US2012/052036

§ 371 Date: Nov. 14, 2014

Related U.S. Application Data
(63) Continuation-in-part of application No. 13/592,827, filed on Aug. 23, 2012.
(60) Provisional application No. 61/527,922, filed on Aug. 26, 2011.
(60) Provisional application No. 61/532,825, filed on Sep. 9, 2011.
(60) Provisional application No. 61/527,922, filed on Aug. 26, 2011.
(60) Provisional application No. 61/532,825, filed on Sep. 9, 2011.

(51) Int. Cl. C12N 15/10

(52) U.S. Cl. CPC C12N 15/1031; C12N 15/1093

(57) ABSTRACT

Aspects of the invention relate to methods, compositions and algorithms for designing and producing a target nucleic acid. The method can include: (1) providing a plurality of blunt-end double-stranded nucleic acid fragments having a restriction enzyme recognition sequence at both ends thereof; (2) producing via enzymatic digestion a plurality of cohesive-end double-stranded nucleic acid fragments each having two different and non-complementary overhangs; (3) ligating the plurality of cohesive-end double-stranded nucleic acid fragments with a ligase; and (4) forming a linear arrangement of the plurality of cohesive-end double-stranded nucleic acid fragments, wherein the unique arrangement comprises the target nucleic acid. In certain embodiments, the plurality of blunt-end double-stranded nucleic acid fragments can be provided by: releasing a plurality of oligonucleotides synthesized on a solid support; and synthesizing complementary strands of the plurality of oligonucleotides using a polymerase based reaction.

### RELATED APPLICATIONS

This application claims the benefit of and priority to U.S. patent application Ser. No. 13/592,827 filed Aug. 23, 2012, U.S. Provisional Application No. 61/527,922, filed Aug. 26, 2011, and U.S. Provisional Application No. 61/532,825, filed Sep. 9, 2011, each of which is incorporated herein by reference in its entirety.

### FIELD OF THE INVENTION

Methods and compositions of the invention relate to nucleic acid assembly, and particularly to high fidelity, multiplex nucleic acid assembly reactions.

### BACKGROUND

Recombinant and synthetic nucleic acids have many applications in research, industry, agriculture, and medicine. Recombinant and synthetic nucleic acids can be used to express and obtain large amounts of polypeptides, including enzymes, antibodies, growth factors, receptors, and other polypeptides that may be used for a variety of medical, industrial, or agricultural purposes. Recombinant and synthetic nucleic acids also can be used to produce genetically modified organisms including modified bacteria, yeast, mammals, plants, and other organisms. Genetically modified organisms may be used in research (e.g., as animal models of disease, as tools for understanding biological processes, etc.), in industry (e.g., as host organisms for protein expression, as bioreactors for generating industrial products, as tools for environmental remediation, for isolating or modifying natural compounds with industrial applications, etc.), in agriculture (e.g., modified crops with increased yield or increased resistance to disease or environmental stress, etc.), and for other applications. Recombinant and synthetic nucleic acids also may be used as therapeutic compositions (e.g., for modifying gene expression, for gene therapy, etc.) or as diagnostic tools (e.g., as probes for disease conditions, etc.).

Numerous techniques have been developed for modifying existing nucleic acids (e.g., naturally occurring nucleic acids) to generate recombinant nucleic acids. For example, combinations of nucleic acid amplification, mutagenesis, nuclease digestion, ligation, cloning and other techniques may be used to produce many different recombinant nucleic acids. Chemically synthesized polynucleotides are often used as primers or adaptors for nucleic acid amplification, mutagenesis, and cloning.

Techniques also are being developed for de novo nucleic acid assembly whereby nucleic acids are made (e.g., chemically synthesized) and assembled to produce longer target nucleic acids of interest. For example, different multiplex assembly techniques are being developed for assembling oligonucleotides into larger synthetic nucleic acids that can be used in research, industry, agriculture, and/or medicine. However, one limitation of currently available assembly techniques is the relatively high error rate. As such, high fidelity, low cost assembly methods are needed.

### SUMMARY OF THE INVENTION

Aspects of the invention relate to methods of producing a target nucleic acid. The method, according to some embodiments, includes: (1) providing a plurality of blunt-end double-stranded nucleic acid fragments having a restriction enzyme recognition sequence at both ends of each of the plurality of blunt-end double-stranded nucleic acid fragments; (2) producing a plurality of cohesive-end double-stranded nucleic acid fragments via enzymatic digestion of the plurality of blunt-end double-stranded nucleic acid fragments in proximity of the restriction enzyme recognition sequence, wherein each of the plurality of cohesive-end double-stranded nucleic acid fragments have two different and non-complementary overhangs; (3) ligating the plurality of cohesive-end double-stranded nucleic acid fragments with a ligase, wherein a first overhang of a first cohesive-end double-stranded nucleic acid fragment is uniquely complementary to a second overhang of a second cohesive-end double-stranded nucleic acid fragment; and (4) forming a linear arrangement of the plurality of cohesive-end double-stranded nucleic acid fragments, wherein the unique arrangement comprises the target nucleic acid. In certain embodiments, the plurality of blunt-end double-stranded nucleic acid fragments can be provided by releasing a plurality of oligonucleotides synthesized on a solid support, and synthesizing complementary strands of the plurality of oligonucleotides using a polymerase based reaction.

In another aspect of the invention, a method for designing a plurality of starting nucleic acids to be assembled into a target nucleic acid is provided. The method, according to some embodiments, can include: (1) obtaining a target sequence of a target nucleic acid; (2) selecting a plurality of subsequences therein such that every two adjacent subsequences overlap with each other by N bases; (3) storing the resulting overlapping N-base sequences in a memory; (4) comparing the overlapping N-base sequences to one another to ensure that they differ from one another by at least one base; and (5) repeating steps (2) to (4) until a plurality of satisfactory starting nucleic acids are obtained wherein any two adjacent starting nucleic acids uniquely overlap with each other by N bases.

Yet another aspect of the invention relates to a plurality of starting nucleic acids to be assembled into a target nucleic acid, designed according to the methods described herein. In certain embodiments, the plurality of starting nucleic acids can each further include an engineered universal primer binding site for amplifying the plurality of starting nucleic acids therefrom. The plurality of starting nucleic acids can also each further include an engineered restriction enzyme recognition sequence.

In still another aspect, a system for assembling a target nucleic acid is provided. The system includes: (1) a solid support for synthesizing the plurality of starting nucleic acids described herein, wherein each starting nucleic acid further comprises an engineered universal primer binding site and an engineered restriction enzyme recognition sequence; (2) a polymerase reaction unit for synthesizing complementary strands of the plurality of starting nucleic acids a polymerase based reaction using a universal primer complementary to the universal primer binding site, thereby producing a plurality of blunt-end double-stranded nucleic acid fragments; (3) a digestion unit for producing a plurality of cohesive-end double-stranded nucleic acid fragments via enzymatic digestion of the plurality of blunt-end double-stranded nucleic acid fragments in proximity of the restriction enzyme recognition sequence, wherein the plurality of cohesive-end double-stranded nucleic acid fragments each have two different and non-complementary overhangs; and (4) a ligation unit for ligating the plurality of cohesive-end double-stranded nucleic acid fragments with a ligase, wherein a first overhang of a first cohesive-end double-stranded nucleic acid fragment is uniquely complementary to a second overhang of a second cohesive-end double-stranded nucleic acid fragment.

A further aspect of the invention provides a computer program product for designing a plurality of starting nucleic acids to be assembled into a target nucleic acid, said program residing on a hardware computer readable storage medium and having a plurality of instructions which, when executed by a processor, cause the processor to perform operations comprising: (1) obtaining a target sequence of a target nucleic acid; (2) selecting a plurality of subsequences therein such that every two adjacent subsequences overlap with each other by N bases; (3) storing the resulting overlapping N-base sequences in a memory; (4) comparing the overlapping N-base sequences to one another to ensure that they differ from one another by at least one base; and (5) repeating steps (2) to (4) until a plurality of satisfactory starting nucleic acids are obtained wherein any two adjacent starting nucleic acids uniquely overlap with each other by N bases.

### BRIEF DESCRIPTION OF THE FIGURES

FIG. 1 illustrates an exemplary design of oligonucleotides for a multiplex oligonucleotide assembly reaction.

FIG. 2 illustrates relative position of primers used for testing products from the multiplex assembly reaction.

FIG. 3 illustrates an embodiment of a pairwise oligonucleotide assembly reaction.

FIG. 4 illustrates embodiments of a multiplex oligonucleotide assembly reaction.

FIG. 5 illustrates a PCR based test of the products of the multiplex oligonucleotide assembly reaction of FIG. 4.

FIG. 6 illustrates sequencing confirmation of the products of the multiplex oligonucleotide assembly reaction of FIG. 4.

FIGS. 7A and 7B illustrate embodiments of a pairwise mismatch ligation assay.

FIG. 8 illustrates alternative assembly products based on the design of FIG. 1.

FIGS. 9A and 9B illustrate two design strategies for sequences flanking assembly fragments.

FIGS. 10A and 10B illustrate two offset assembly strategies.

### DETAILED DESCRIPTION OF THE INVENTION

Aspects of the invention relate to methods and compositions for covalently joining a plurality of nucleic acid fragments to produce a longer nucleic acid product in a single assembly step. Aspects of the invention can be used to assemble large numbers of nucleic acid fragments efficiently, and/or to reduce the number of steps required to generate large nucleic acid products, while reducing assembly error rate. Aspects of the invention can be incorporated into nucleic assembly procedures to increase assembly fidelity, throughput and/or efficiency, decrease cost, and/or reduce assembly time. In some embodiments, aspects of the invention may be automated and/or implemented in a high throughput assembly context to facilitate parallel production of many different target nucleic acid products.

### Multiplex Oligonucleotide Assembly

A predetermined nucleic acid fragment may be assembled from a plurality of different starting nucleic acids (e.g., oligonucleotides) in a multiplex assembly reaction (e.g., a multiplex enzyme-mediated reaction, a multiplex chemical assembly reaction, or a combination thereof). Certain aspects of multiplex nucleic acid assembly reactions are illustrated by the following description of certain embodiments of multiplex oligonucleotide assembly reactions. It should be appreciated that the description of the assembly reactions in the context of oligonucleotides is not intended to be limiting. The assembly reactions described herein may be performed using starting nucleic acids obtained from one or more different sources (e.g., synthetic or natural polynucleotides, nucleic acid amplification products, nucleic acid degradation products, oligonucleotides, etc.). The starting nucleic acids may be referred to as assembly nucleic acids (e.g., assembly oligonucleotides). As used herein, an assembly nucleic acid has a sequence that is designed to be incorporated into the nucleic acid product generated during the assembly process. However, it should be appreciated that the description of the assembly reactions in the context of double-stranded nucleic acids is not intended to be limiting. In some embodiments, one or more of the starting nucleic acids illustrated in the figures and described herein may be provided as single-stranded nucleic acids. Accordingly, it should be appreciated that where the figures and description illustrate the assembly of cohesive-end double-stranded nucleic acids, the presence of one or more single-stranded nucleic acids is contemplated.

As used herein, an oligonucleotide may be a nucleic acid molecule comprising at least two covalently bonded nucleotide residues. In some embodiments, an oligonucleotide may be between 10 and 1,000 nucleotides long. For example, an oligonucleotide may be between 10 and 500 nucleotides long, or between 500 and 1,000 nucleotides long. In some embodiments, an oligonucleotide may be between about 20 and about 300 nucleotides long (e.g., from about 30 to 250, 40 to 220, 50 to 200, 60 to 180, or about 65 or about 150 nucleotides long), between about 100 and about 200, between about 200 and about 300 nucleotides, between about 300 and about 400, or between about 400 and about 500 nucleotides long. However, shorter or longer oligonucleotides may be used. An oligonucleotide may be a single-stranded nucleic acid. However, in some embodiments a double-stranded oligonucleotide may be used as described herein. In certain embodiments, an oligonucleotide may be chemically synthesized as described in more detail below. In some embodiments, an input nucleic acid (e.g., synthetic oligonucleotide) may be amplified before use. The resulting product may be double-stranded.

In certain embodiments, each oligonucleotide may be designed to have a sequence that is identical to a different portion of the sequence of a predetermined target nucleic acid that is to be assembled. Accordingly, in some embodiments each oligonucleotide may have a sequence that is identical to a portion of one of the two strands of a double-stranded target nucleic acid. For clarity, the two complementary strands of a double stranded nucleic acid are referred to herein as the positive (P) and negative (N) strands. This designation is not intended to imply that the strands are sense and anti-sense strands of a coding sequence. They refer only to the two complementary strands of a nucleic acid (e.g., a target nucleic acid, an intermediate nucleic acid fragment, etc.) regardless of the sequence or function of the nucleic acid. Accordingly, in some embodiments a P strand may be a sense strand of a coding sequence, whereas in other embodiments a P strand may be an anti-sense strand of a coding sequence. It should be appreciated that the reference to complementary nucleic acids or complementary nucleic acid regions herein refers to nucleic acids or regions thereof that have sequences which are reverse complements of each other so that they can hybridize in an antiparallel fashion typical of natural DNA.

According to one aspect of the invention, a target nucleic acid may be either the P strand, the N strand, or a double-stranded nucleic acid comprising both the P and N strands. It should be appreciated that different oligonucleotides may be designed to have different lengths.

In some embodiments, one or more different oligonucleotides may have overlapping sequence regions (e.g., overlapping 5′ regions and/or overlapping 3′ regions). Overlapping sequence regions may be identical (i.e., corresponding to the same strand of the nucleic acid fragment) or complementary (i.e., corresponding to complementary strands of the nucleic acid fragment). The plurality of oligonucleotides may include one or more oligonucleotide pairs with overlapping identical sequence regions, one or more oligonucleotide pairs with overlapping complementary sequence regions, or a combination thereof. Overlapping sequences may be of any suitable length. For example, overlapping sequences may encompass the entire length of one or more nucleic acids used in an assembly reaction. Overlapping sequences may be between about 2 and about 50 (e.g., between 3 and 20, between 3 and 10, between 3 and 8, or 4, 5, 6, 7, 8, 9, etc. nucleotides long). However, shorter, longer or intermediate overlapping lengths may be used. It should be appreciated that overlaps between different input nucleic acids used in an assembly reaction may have different lengths and/or sequences. For example, the overlapping sequences may be different than one another by at least one nucleotide, 2 nucleotides, 3 nucleotides, or more. Assuming that the overlapping sequences differ from one another by x nucleotides, then up to (4^{x}+1) pieces of different input nucleic acids can be assembled together in one reaction.

In a multiplex oligonucleotide assembly reaction designed to generate a predetermined nucleic acid fragment, the combined sequences of the different oligonucleotides in the reaction may span the sequence of the entire nucleic acid fragment on either the positive strand, the negative strand, both strands, or a combination of portions of the positive strand and portions of the negative strand. The plurality of different oligonucleotides may provide either positive sequences, negative sequences, or a combination of both positive and negative sequences corresponding to the entire sequence of the nucleic acid fragment to be assembled. In some embodiments, the plurality of oligonucleotides may include one or more oligonucleotides having sequences identical to one or more portions of the positive sequence, and one or more oligonucleotides having sequences that are identical to one or more portions of the negative sequence of the nucleic acid fragment. One or more pairs of different oligonucleotides may include sequences that are identical to overlapping portions of the predetermined nucleic acid fragment sequence as described herein (e.g., overlapping sequence portions from the same or from complementary strands of the nucleic acid fragment). In some embodiments, the plurality of oligonucleotides includes a set of oligonucleotides having sequences that combine to span the entire positive sequence and a set oligonucleotides having sequences that combine to span the entire negative sequence of the predetermined nucleic acid fragment. However, in certain embodiments, the plurality of oligonucleotides may include one or more oligonucleotides with sequences that are identical to sequence portions on one strand (either the positive or negative strand) of the nucleic acid fragment, but no oligonucleotides with sequences that are complementary to those sequence portions. In one embodiment, a plurality of oligonucleotides includes only oligonucleotides having sequences identical to portions of the positive sequence of the predetermined nucleic acid fragment. In one embodiment, a plurality of oligonucleotides includes only oligonucleotides having sequences identical to portions of the negative sequence of the predetermined nucleic acid fragment. These oligonucleotides may be assembled by sequential ligation or in an extension-based reaction (e.g., if an oligonucleotide having a 3′ region that is complementary to one of the plurality of oligonucleotides is added to the reaction).

In one aspect, a nucleic acid fragment may be assembled in a ligase-mediated assembly reaction from a plurality of oligonucleotides that are combined and ligated in one or more rounds of ligase-mediated ligations. Ligase-based assembly techniques may involve one or more suitable ligase enzymes that can catalyze the covalent linking of adjacent 3′ and 5′ nucleic acid termini (e.g., a 5′ phosphate and a 3′ hydroxyl of nucleic acid(s) annealed on a complementary template nucleic acid such that the 3′ terminus is immediately adjacent to the 5′ terminus). Accordingly, a ligase may catalyze a ligation reaction between the 5′ phosphate of a first nucleic acid to the 3′ hydroxyl of a second nucleic acid if the first and second nucleic acids are annealed next to each other on a template nucleic acid). A ligase may be obtained from recombinant or natural sources. In some embodiments, one or more low temperature (e.g., room temperature or lower) ligases may be used (e.g., T3 DNA ligase, T4 DNA ligase, T7 DNA ligase, and/or E. coli DNA Ligase). A lower temperature ligase may be useful for shorter overhangs (e.g., about 3, about 4, about 5, or about 6 base overhangs) that may not be stable at higher temperatures. A ligase may also be a heat-stable ligase. In some embodiments, a thermostable ligase from a thermophilic organism may be used. Examples of thermostable DNA ligases include, but are not limited to: Tth DNA ligase (from Thermus thermophilics, available from, for example, Eurogentec and GeneCraft); Pfu DNA ligase (a hyperthermophilic ligase from Pyrococcus furiosus); Taq ligase (from Thermus aquaticus), any other suitable heat-stable ligase, or any combination thereof.

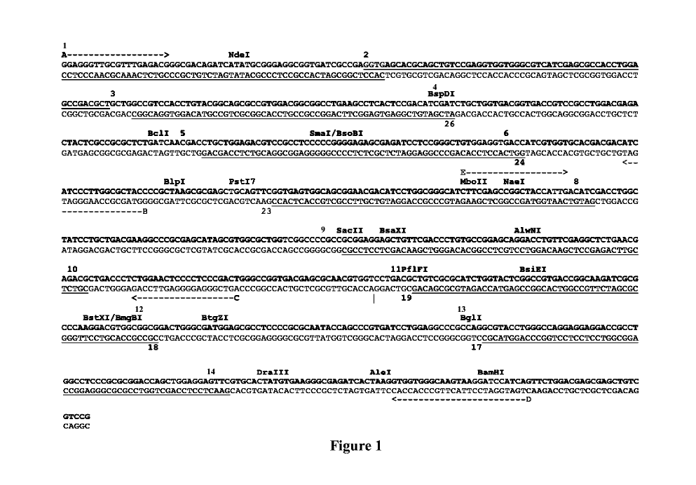

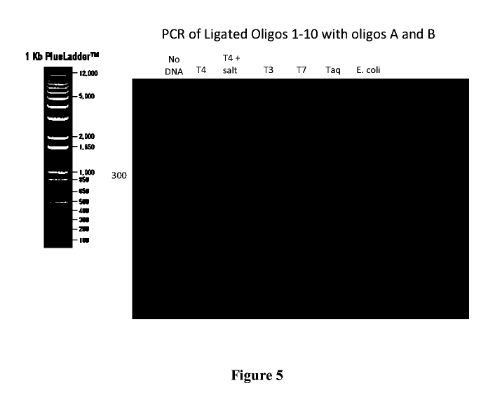

Aspects of the invention may be used to enhance different types of nucleic acid assembly reactions (e.g., multiplex nucleic acid assembly reactions). Aspects of the invention may be used in combination with one or more assembly reactions described in, for example, Carr et al., 2004, Nucleic Acids Research, Vol. 32, No 20, e162 (9 pages); Richmond et al., 2004, Nucleic Acids Research, Vol. 32, No 17, pp. 5011-5018; Caruthers et al., 1972, J. Mol. Biol. 72, 475-492; Hecker et al., 1998, Biotechniques 24:256-260; Kodumal et al., 2004, PNAS Vol. 101, No. 44, pp. 15573-15578; Tian et al., 2004, Nature, Vol. 432, pp. 1050-1054; and U.S. Pat. Nos. 6,008,031 and 5,922,539, the disclosures of which are incorporated herein by reference. Certain embodiments of multiplex nucleic acid assembly reactions for generating a predetermined nucleic acid fragment are illustrated with reference to FIGS. 1-10. It should be appreciated that synthesis and assembly methods described herein (including, for example, oligonucleotide synthesis, step-wise assembly, multiplex nucleic acid assembly, hierarchical assembly of nucleic acid fragments, or any combination thereof) may be performed in any suitable format, including in a reaction tube, in a multi-well plate, on a surface, on a column, in a microfluidic device (e.g., a microfluidic tube), a capillary tube, etc. For example, some embodiments, the target nucleic acid can be assembled by “recursive assembly” or “hierarchical assembly.” In this embodiment, the target nucleic acid is divided first into two or more overlapping nucleic acid fragments (or subassembly fragments). Each nucleic acid fragments is then subdivided into two or more overlapping smaller nucleic acid fragments.

### Synthetic Oligonucleotides

Oligonucleotides may be synthesized using any suitable technique. For example, oligonucleotides may be synthesized on a column or other support (e.g., a chip). Examples of chip-based synthesis techniques include techniques used in synthesis devices or methods available from CombiMatrix, Agilent, Affymetrix, or other sources. A synthetic oligonucleotide may be of any suitable size, for example between 10 and 1,000 nucleotides long (e.g., between 10 and 200, 200 and 500, 500 and 1,000 nucleotides long, or any combination thereof). An assembly reaction may include a plurality of oligonucleotides, each of which independently may be between 10 and 300 nucleotides in length (e.g., between 20 and 250, between 30 and 200, 50 to 150, 50 to 100, or any intermediate number of nucleotides). However, one or more shorter or longer oligonucleotides may be used in certain embodiments.

As used herein, the term “support” and “substrate” are used interchangeably and refers to a porous or non-porous solvent insoluble material on which polymers such as nucleic acids are synthesized or immobilized. As used herein “porous” means that the material contains pores having substantially uniform diameters (for example in the nm range). Porous materials can include but are not limited to, paper, synthetic filters and the like. In such porous materials, the reaction may take place within the pores. The support can have any one of a number of shapes, such as pin, strip, plate, disk, rod, bends, cylindrical structure, particle, including bead, nanoparticle and the like. The support can have variable widths.

The support can be hydrophilic or capable of being rendered hydrophilic. The support can include inorganic powders such as silica, magnesium sulfate, and alumina; natural polymeric materials, particularly cellulosic materials and materials derived from cellulose, such as fiber containing papers, e.g., filter paper, chromatographic paper, etc.; synthetic or modified naturally occurring polymers, such as nitrocellulose, cellulose acetate, poly (vinyl chloride), polyacrylamide, cross linked dextran, agarose, polyacrylate, polyethylene, polypropylene, poly (4-methylbutene), polystyrene, polymethacrylate, poly(ethylene terephthalate), nylon, poly(vinyl butyrate), polyvinylidene difluoride (PVDF) membrane, glass, controlled pore glass, magnetic controlled pore glass, ceramics, metals, and the like; either used by themselves or in conjunction with other materials.

In some embodiments, oligonucleotides are synthesized on an array format. For example, single-stranded oligonucleotides are synthesized in situ on a common support wherein each oligonucleotide is synthesized on a separate or discrete feature (or spot) on the substrate. In preferred embodiments, single-stranded oligonucleotides are bound to the surface of the support or feature. As used herein, the term “array” refers to an arrangement of discrete features for storing, routing, amplifying and releasing oligonucleotides or complementary oligonucleotides for further reactions. In a preferred embodiment, the support or array is addressable: the support includes two or more discrete addressable features at a particular predetermined location (i.e., an “address”) on the support. Therefore, each oligonucleotide molecule of the array is localized to a known and defined location on the support. The sequence of each oligonucleotide can be determined from its position on the support. Moreover, addressable supports or arrays enable the direct control of individual isolated volumes such as droplets. The size of the defined feature can be chosen to allow formation of a microvolume droplet on the feature, each droplet being kept separate from each other. As described herein, features are typically, but need not be, separated by interfeature spaces to ensure that droplets between two adjacent features do not merge. Interfeatures will typically not carry any oligonucleotide on their surface and will correspond to inert space. In some embodiments, features and interfeatures may differ in their hydrophilicity or hydrophobicity properties. In some embodiments, features and interfeatures may comprise a modifier as described herein.

Arrays may be constructed, custom ordered or purchased from a commercial vendor (e.g., CombiMatrix, Agilent, Affymetrix, Nimblegen). Oligonucleotides are attached, spotted, immobilized, surface-bound, supported or synthesized on the discrete features of the surface or array. Oligonucleotides may be covalently attached to the surface or deposited on the surface. Various methods of construction are well known in the art, e.g., maskless array synthesizers, light directed methods utilizing masks, flow channel methods, spotting methods etc.

In some embodiments, construction and/or selection oligonucleotides may be synthesized on a solid support using maskless array synthesizer (MAS). Maskless array synthesizers are described, for example, in PCT application No. WO 99/42813 and in corresponding U.S. Pat. No. 6,375,903. Other examples are known of maskless instruments which can fabricate a custom DNA microarray in which each of the features in the array has a single-stranded DNA molecule of desired sequence.

Other methods for synthesizing construction and/or selection oligonucleotides include, for example, light-directed methods utilizing masks, flow channel methods, spotting methods, pin-based methods, and methods utilizing multiple supports.

Light directed methods utilizing masks (e.g., VLSIPS™ methods) for the synthesis of oligonucleotides is described, for example, in U.S. Pat. Nos. 5,143,854; 5,510,270 and 5,527,681. These methods involve activating predefined regions of a solid support and then contacting the support with a preselected monomer solution. Selected regions can be activated by irradiation with a light source through a mask much in the manner of photolithography techniques used in integrated circuit fabrication. Other regions of the support remain inactive because illumination is blocked by the mask and they remain chemically protected. Thus, a light pattern defines which regions of the support react with a given monomer. By repeatedly activating different sets of predefined regions and contacting different monomer solutions with the support, a diverse array of polymers is produced on the support. Other steps, such as washing unreacted monomer solution from the support, can be optionally used. Other applicable methods include mechanical techniques such as those described in U.S. Pat. No. 5,384,261.

Additional methods applicable to synthesis of construction and/or selection oligonucleotides on a single support are described, for example, in U.S. Pat. No. 5,384,261. For example, reagents may be delivered to the support by either (1) flowing within a channel defined on predefined regions or (2) “spotting” on predefined regions. Other approaches, as well as combinations of spotting and flowing, may be employed as well. In each instance, certain activated regions of the support are mechanically separated from other regions when the monomer solutions are delivered to the various reaction sites. Flow channel methods involve, for example, microfluidic systems to control synthesis of oligonucleotides on a solid support. For example, diverse polymer sequences may be synthesized at selected regions of a solid support by forming flow channels on a surface of the support through which appropriate reagents flow or in which appropriate reagents are placed. Spotting methods for preparation of oligonucleotides on a solid support involve delivering reactants in relatively small quantities by directly depositing them in selected regions. In some steps, the entire support surface can be sprayed or otherwise coated with a solution, if it is more efficient to do so. Precisely measured aliquots of monomer solutions may be deposited dropwise by a dispenser that moves from region to region.

Pin-based methods for synthesis of oligonucleotides on a solid support are described, for example, in U.S. Pat. No. 5,288,514. Pin-based methods utilize a support having a plurality of pins or other extensions. The pins are each inserted simultaneously into individual reagent containers in a tray. An array of 96 pins is commonly utilized with a 96-container tray, such as a 96-wells microtiter dish. Each tray is filled with a particular reagent for coupling in a particular chemical reaction on an individual pin. Accordingly, the trays will often contain different reagents. Since the chemical reactions have been optimized such that each of the reactions can be performed under a relatively similar set of reaction conditions, it becomes possible to conduct multiple chemical coupling steps simultaneously.

Other suitable microarrays and methods for synthesizing oligonucleotides include those described in U.S. Pat. Nos. 7,323,320 and 7,563,600, the entire disclosures of which are hereby incorporated herein by reference in their entirety. In an example, the oligonucleotides synthesized therefrom are chemically, enzymatically, or physically cleaved or otherwise released from the microarrays for further amplification, restriction enzyme digestion and/or assembly.

In another embodiment, a plurality of oligonucleotides may be synthesized or immobilized (e.g. attached) on multiple supports, such as beads. One example is a bead based synthesis method which is described, for example, in U.S. Pat. Nos. 5,770,358; 5,639,603; and 5,541,061. For the synthesis of molecules such as oligonucleotides on beads, a large plurality of beads is suspended in a suitable carrier (such as water) in a container. The beads are provided with optional spacer molecules having an active site to which is complexed, optionally, a protecting group. At each step of the synthesis, the beads are divided for coupling into a plurality of containers. After the nascent oligonucleotide chains are deprotected, a different monomer solution is added to each container, so that on all beads in a given container, the same nucleotide addition reaction occurs. The beads are then washed of excess reagents, pooled in a single container, mixed and re-distributed into another plurality of containers in preparation for the next round of synthesis. It should be noted that by virtue of the large number of beads utilized at the outset, there will similarly be a large number of beads randomly dispersed in the container, each having a unique oligonucleotide sequence synthesized on a surface thereof after numerous rounds of randomized addition of bases. An individual bead may be tagged with a sequence which is unique to the double-stranded oligonucleotide thereon, to allow for identification during use.

In yet another embodiment, a plurality of oligonucleotides may be attached or synthesized on nanoparticles. Nanoparticles includes but are not limited to metal (e.g., gold, silver, copper and platinum), semiconductor (e.g., CdSe, CdS, and CdS coated with ZnS) and magnetic (e.g., ferromagnetite) colloidal materials. Methods to attach oligonucleotides to the nanoparticles are known in the art. In another embodiment, nanoparticles are attached to the substrate. Nanoparticles with or without immobilized oligonucleotides can be attached to substrates as described in, e.g., Grabar et al., Analyt. Chem., 67, 73-743 (1995); Bethell et al., J. Electroanal. Chem., 409, 137 (1996); Bar et al., Langmuir, 12, 1172 (1996); Colvin et al., J. Am. Chem. Soc., 114, 5221 (1992). Naked nanoparticles may be first attached to the substrate and oligonucleotides can be attached to the immobilized nanoparticles.

Pre-synthesized oligonucleotide and/or polynucleotide sequences may be attached to a support or synthesized in situ using light-directed methods, flow channel and spotting methods, inkjet methods, pin-based methods and bead-based methods set forth in the following references: McGall et al. (1996) Proc. Natl. Acad. Sci. U.S.A. 93:13555; Synthetic DNA Arrays In Genetic Engineering, Vol. 20:111, Plenum Press (1998); Duggan et al. (1999) Nat. Genet. S21:10 ; Microarrays: Making Them and Using Them In Microarray Bioinformatics, Cambridge University Press, 2003; U.S. Patent Application Publication Nos. 2003/0068633 and 2002/0081582; U.S. Pat. Nos. 6,833,450, 6,830,890, 6,824,866, 6,800,439, 6,375,903 and 5,700,637; and PCT Publication Nos. WO 04/031399, WO 04/031351, WO 04/029586, WO 03/100012, WO 03/066212, WO 03/065038, WO 03/064699, WO 03/064027, WO 03/064026, WO 03/046223, WO 03/040410 and WO 02/24597; the disclosures of which are incorporated herein by reference in their entirety for all purposes. In some embodiments, pre-synthesized oligonucleotides are attached to a support or are synthesized using a spotting methodology wherein monomers solutions are deposited dropwise by a dispenser that moves from region to region (e.g., ink jet). In some embodiments, oligonucleotides are spotted on a support using, for example, a mechanical wave actuated dispenser.

A preparation of an oligonucleotide designed to have a certain sequence may include oligonucleotide molecules having the designed sequence in addition to oligonucleotide molecules that contain errors (e.g., that differ from the designed sequence at least at one position). A sequence error may include one or more nucleotide deletions, additions, substitutions (e.g., transversion or transition), inversions, duplications, or any combination of two or more thereof. Oligonucleotide errors may be generated during oligonucleotide synthesis. Different synthetic techniques may be prone to different error profiles and frequencies. In some embodiments, error rates may vary from 1/10 to 1/200 errors per base depending on the synthesis protocol that is used. However, in some embodiments, lower error rates may be achieved. Also, the types of errors may depend on the synthetic techniques that are used. For example, in some embodiments chip-based oligonucleotide synthesis may result in relatively more deletions than column-based synthetic techniques.

In some embodiments, one or more oligonucleotide preparations may be subjected to an error reduction or error filtration process to remove (or reduce the number or the frequency of) error-containing oligonucleotides. Such process can be used to increase the number of error-free oligonucleotides in the oligonucleotide preparations. Methods for conducting error reduction or error filtration can include, for example, hybridization to a selection oligonucleotide, binding to a mismatch binding agent or to a mismatch binding protein or combinations thereof.

In some embodiments, a hybridization technique may be used wherein an oligonucleotide preparation (i.e. construction oligonucleotides) is hybridized under stringent conditions, one or more times, to an immobilized oligonucleotide preparation (i.e. selection oligonucleotides) designed to have a complementary sequence. The term “selection oligonucleotide” as used herein refers to a single-stranded oligonucleotide that is complementary to at least a portion of a construction oligonucleotide (or the complement of the construction oligonucleotide). Selection oligonucleotides may be used for removing copies of a construction oligonucleotide that contain sequencing errors (e.g., a deviation from the desired sequence) from a pool of construction oligonucleotides. In some embodiments, a selection oligonucleotide may be end immobilized on a substrate. Yet in other embodiments, the selection oligonucleotides can be in solution. In one embodiment, selection oligonucleotides can be synthetic oligonucleotides that have been synthesized in parallel on a substrate as disclosed herein.

Construction oligonucleotides that do not bind or that form unstable duplexes may be removed in order to selectively or specifically remove error-containing oligonucleotides that would destabilize hybridization under the conditions used. It should be appreciated that this process may not remove all error-containing oligonucleotides since some error-containing oligonucleotides may still bind to the immobilized selection oligonucleotides with sufficient affinity through this selection process. For example, the error-containing oligonucleotides may differ from the selection oligonucleotide by one or two bases and may still bind to the selection oligonucleotides under the selection process reaction conditions.

In some embodiments, a nucleic acid binding protein or recombinase (e.g., RecA) may be included in one or more of the oligonucleotide processing steps to improve the selection of error-free oligonucleotides. For example, by preferentially promoting the hybridization of oligonucleotides that are completely complementary with the immobilized oligonucleotides, the amount of error-containing oligonucleotides that are bound may be reduced. As a result, the oligonucleotide processing procedure described herein may remove more error-containing oligonucleotides and generate an oligonucleotide preparation that has a lower error frequency (e.g., with an error rate of less than 1/50, less than 1/100, less than 1/200, less than 1/300, less than 1/400, less than 1/500, less than 1/1,000, or less than 1/2,000 errors per base).

In some embodiments, error correction may be included between each process repetition and at the end of the synthesis process to increase the relative population of synthesized polynucleotides without deviation from the desired sequences. Such error correction may include direct sequencing and/or the application of error correction based on correcting enzymes, such as error correcting nucleases (e.g. CEL I), error correction based on MutS or MutS homologs binding or other mismatch binding proteins (see, e.g., International Application No. PCT/US2010/057405), other means of error correction as known in the art or any combination thereof. In an exemplary embodiment, CEL I may be added to the oligonucleotide duplexes in the fluid medium. CEL I is a mismatch specific endonuclease that cleaves all types of mismatches such as single nucleotide polymorphisms, small insertions or deletions. Addition of the endonuclease results in the cleavage of the double-stranded oligonucleotides at the site or region of the mismatch.

It should be appreciated that one or more nucleic acid binding proteins or recombinases are preferably not included in a post-synthesis fidelity optimization technique (e.g., a screening technique using a MutS or MutS homolog), because the optimization procedure involves removing error-containing nucleic acids via the production and removal of heteroduplexes. Accordingly, any nucleic acid binding proteins or recombinases (e.g., RecA) that were included in the synthesis steps is preferably removed (e.g., by inactivation, column purification or other suitable technique) after synthesis and prior to fidelity optimization.

In certain embodiments, it may be helpful to include one or more modified oligonucleotides. An oligonucleotide may be modified by incorporating a modified-base (e.g., a nucleotide analog) during synthesis, by modifying the oligonucleotide after synthesis, or any combination thereof. Examples of modifications include, but are not limited to, one or more of the following: universal bases such as nitro indoles, dP and dK, inosine, uracil; halogenated bases such as BrdU; fluorescent labeled bases; non-radioactive labels such as biotin (as a derivative of dT) and digoxigenin (DIG); 2,4-Dinitrophenyl (DNP); radioactive nucleotides; post-coupling modification such as dR-NH2 (deoxyribose-NEb); Acridine (6-chloro-2-methoxiacridine); and spacer phosphoramides which are used during synthesis to add a spacer “arm” into the sequence, such as C3, C8 (octanediol), C9, C12, HEG (hexaethlene glycol) and C18.

### Amplifying Oligonucleotides

Oligonucleotides may be provided or synthesized as single-stranded synthetic products. In some embodiments, oligonucleotides may also be provided or synthesized as double-stranded preparations including an annealed complementary strand. Oligonucleotides may be molecules of DNA, RNA, PNA, or any combination thereof. A double-stranded oligonucleotide may be produced by amplifying a single-stranded synthetic oligonucleotide or other suitable template (e.g., a sequence in a nucleic acid preparation such as a nucleic acid vector or genomic nucleic acid). Accordingly, a plurality of oligonucleotides designed to have the sequence features described herein may be provided as a plurality of single-stranded oligonucleotides having those feature, or also may be provided along with complementary oligonucleotides. In some embodiments, an oligonucleotide may be phosphorylated (e.g., with a 5′ phosphate). In some embodiments, an oligonucleotide may be non-phosphorylated.

In some embodiments, an oligonucleotide may be amplified using an appropriate primer pair with one primer corresponding to each end of the oligonucleotide (e.g., one that is complementary to the 3′ end of the oligonucleotide and one that is identical to the 5′ end of the oligonucleotide). In some embodiments, an oligonucleotide may be designed to contain a central assembly sequence (designed to be incorporated into the target nucleic acid) flanked by a 5′ amplification sequence (e.g., a 5′ universal sequence) and/or a 3′ amplification sequence (e.g., a 3′ universal sequence). Amplification primers (e.g., between 10 and 50 nucleotides long, between 15 and 45 nucleotides long, about 25 nucleotides long, etc.) corresponding to the flanking amplification sequences may be used to amplify the oligonucleotide (e.g., one primer may be complementary to the 3′ amplification sequence and one primer may have the same sequence as the 5′ amplification sequence). The amplification sequences then may be removed from the amplified oligonucleotide using any suitable technique to produce an oligonucleotide that contains only the assembly sequence.

In some embodiments, a plurality of different oligonucleotides (e.g., about 5, 10, 50, 100, or more) with different central assembly sequences may have identical 5′ amplification sequences and/or identical 3′ amplification sequences. These oligonucleotides can all be amplified in the same reaction using the same amplification primers.

A plurality of oligonucleotides used in an assembly reaction may contain preparations of synthetic oligonucleotides, single-stranded oligonucleotides, double-stranded oligonucleotides, amplification products, oligonucleotides that are processed to remove (or reduce the frequency of) error-containing variants, etc., or any combination of two or more thereof. In some aspects, double-stranded amplification products may be used as assembly oligonucleotides and added to an assembly reaction as described herein. In some embodiments, the oligonucleotide may be amplified while it is still attached to the support. In some embodiments, the oligonucleotide may be removed or cleaved from the support prior to amplification or after amplification.

In some embodiments, a synthetic oligonucleotide may include a central assembly sequence flanked by 5′ and 3′ amplification sequences. The central assembly sequence is designed for incorporation into an assembled target nucleic acid or target subassembly. The flanking sequences are designed for amplification and are not intended to be incorporated into the assembled nucleic acid. The flanking amplification sequences may be used as universal primer sequences to amplify a plurality of different assembly oligonucleotides that share the same amplification sequences but have different central assembly sequences. In some embodiments, the flanking sequences are removed after amplification to produce an oligonucleotide that contains only the assembly sequence.

In certain embodiments, the double-stranded amplification products may be subject to restriction enzyme digestion to remove the flanking sequences. To that end, the flanking sequences can be designed to include one or more restriction sites or restriction enzyme recognition sites. The restriction site may be present at the 5′ or 3′ end of the amplification sequence as long as the cleavage site is between the flanking sequence to be removed and the central assembly sequence. The restriction site may be included in the amplification sequence (i.e., primer binding site). The restriction site may also be outside the amplification sequence.

After restriction enzyme digestion, the cleaved flanking sequences may be separated and removed using any suitable technique. In some embodiments, the cleaved flanking sequences may be fragments less than about 40, about 35, about 30, about 25, about 20, or about 15 bases long. As such, size dependent separation techniques known in the art may be used, such as differential affinity to silica, size filtration, differential precipitation with PEG (polyethylene glycol) or CTAB (cetyltrimethlyammonium bromide), or any combination thereof, so as to separate the cleaved flanking sequences from the central assembly sequences that can be designed to be longer in size than the flanking sequences.

In some embodiments, the amplification primers may be biotinylated. The resulting amplification products thus also become biotinylated at both ends. Upon restriction enzyme digestion, the cleaved flanking sequences having the biotinylated primers retain the biotin tags, while the central assembly sequences are non-biotinylated. Thus, the cleaved flanking sequences can be affinity purified and removed using streptavidin (e.g., bound to a bead, column, or other surface). In some embodiments, the amplification primers also may be designed to include certain sequence features (e.g., restriction sites) that can be used to remove the primer regions after amplification in order to produce a double-stranded assembly fragment that includes the assembly sequence without the flanking amplification sequences.

### Single-Stranded Overhangs

Certain aspects of the invention involve double-stranded nucleic acids with single-stranded overhangs. Overhangs may be generated using any suitable technique. In some embodiments, a double-stranded nucleic acid fragment (e.g., a fragment assembled in a multiplex assembly) may be digested with an appropriate restriction enzyme to generate a terminal single-stranded overhang. In some embodiments, fragments that are designed to be adjacent to each other in an assembled product may be digested with the same enzyme to expose complementary overhangs. Different enzymes that generate complementary overhangs may also used.

In some embodiments, overhangs may be generated using a type IIS restriction enzyme. Type IIS restriction enzymes are enzymes that bind to a double-stranded nucleic acid at one site, referred to as the recognition site, and make a single double stranded cut outside of the recognition site. The double stranded cut, referred to as the cleavage site, is generally situated 0-20 bases away from the recognition site. The recognition site is generally about 4-8 by long. All type IIS restriction enzymes exhibit at least partial asymmetric recognition. Asymmetric recognition means that 5′→3′ recognition sequences are different for each strand of the nucleic acid. The enzyme activity also shows polarity meaning that the cleavage sites are located on only one side of the recognition site. Thus, there is generally only one double stranded cut corresponding to each recognition site. Cleavage generally produces 1-6 nucleotide single-stranded overhangs, with 5′ or 3′ termini, although some enzymes produce blunt ends. Either cut is useful in the context of the invention, although in some instances those producing single-stranded overhangs are produced. To date, about 80 type IIS enzymes have been identified. Suitable examples include but are not limited to BstF5 I, BtsC I, BsrD I, Bts I, Alw I, Bcc I, BsmA I, Ear I, Mly I (blunt), Ple I, Bmr I, Bsa I, BsmB I, BspQ I, Fau I, MnI I, Sap I, Bbs I, BciV I, Hph I, Mbo II, BfuA I, BspCN I, BspM I, SfaN I, Hga I, BseR I, Bbv I, Eci I, Fok I, BceA I, BsmF I, BtgZ I, BpuE I, Bsg I, Mme I, BseG I, Bse3D I, BseM I, Ac1W I, A1w26 1, Bst6 1, BstMA I, Eaml 104 1, Ksp632 I, Pps 1₅ Sch I (blunt), Bfi I, Bso3l 1, BspTN I, Eco3l I, Esp3 I, Smu I, Bfu I, Bpi I, BpuA I, BstV2 I, AsuHP I, Acc36 I, Lwe I, Aar I, BseM II, TspDT I, TspGW I, BseX I, BstVl I, Eco571₅ Eco57M I₅ Gsu I₅ and Beg I. In some embodiments, Bsa I, BsmB I, BspQ I, BtgZ I, BsmF I, Fok I, Bbv I, any variant thereof, or any combination thereof can be used. Such enzymes and information regarding their recognition and cleavage sites are available from commercial suppliers such as New England Biolabs.

In some embodiments, each of a plurality of nucleic acid fragments designed for assembly may have a type IIS restriction site at each end. The type IIS restriction sites may be oriented so that the cleavage sites are internal relative to the recognition sequences. As a result, enzyme digestion exposes an internal sequence (e.g., an overhang within an internal sequence) and removes the recognition sequences from the ends. Accordingly, the same type IIS sites may be used for both ends of all of the nucleic acid fragments being prepared for assembly. However, different type IIS sites also may be used. Two fragments that are designed to be adjacent in an assembled product each may include an identical overlapping terminal sequence and a flanking type IIS site that is appropriately located to expose complementary overhangs within the overlapping sequence upon restriction enzyme digestion. Accordingly, a plurality of nucleic acid fragments may be generated with different complementary overhangs. The restriction site at each end of a nucleic acid fragment may be located such that digestion with the appropriate type IIS enzyme removes the restriction site and exposes a single-stranded region that is complementary to a single-stranded region on a nucleic acid fragment that is designed to be adjacent in the assembled nucleic acid product. In certain embodiments, restriction enzymes can be selected such that the assembly nucleic acid fragments are free of the corresponding restriction sites.

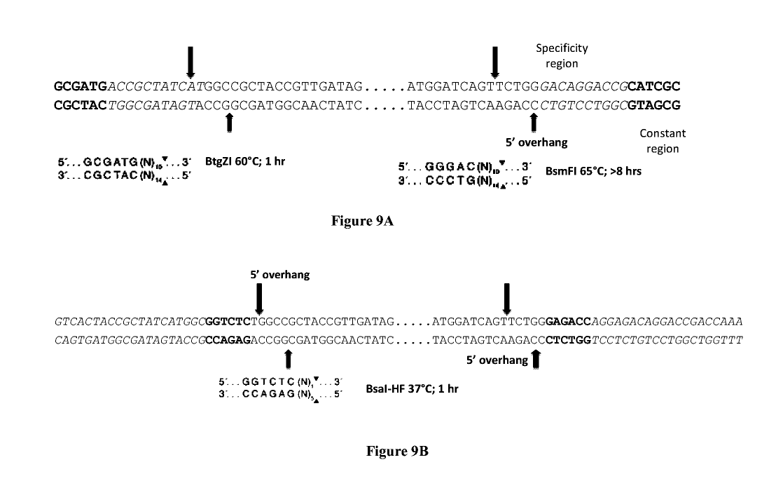

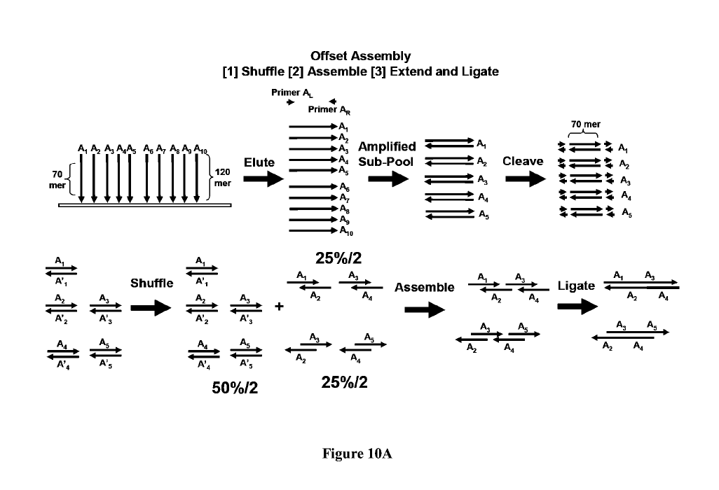

As discussed above, restriction sites can be placed inside or outside, 5′ or 3′ to the amplification sequence. As FIG. 9A illustrates, restriction sites (shown in bold) can be included within the amplification sequence (shown in italic) and distal to the central assembly fragment (black). By way of example, BtgZI and BsmFI sites are used at either end of the double-stranded assembly fragment, and their respective cleavage sites are indicated by arrows. BtgZI and BsmFI both cleave at 10 nucleotides/14 nucleotides away from their recognition sites. Other restriction enzymes that cleave at a short distance (e.g., 5-25, 10-20, or about 15 nucleotides) from the recognition site can also be used. Alternatively, as FIG. 9B illustrates, restriction sites (shown in bold) can be outside the amplification sequence (shown in italic) and proximal to the central assembly fragment (normal font). BsaI sites are used at both ends of the double-stranded assembly fragment as an example, the cleavage sites of which are also indicated by arrows. As can be seen from FIGS. 9A and 9B, when restriction sites are placed distal to the central assembly fragment and included in the amplification sequence, the overall length of the starting nucleic acid is shorter than when restriction sites are placed proximal to the central assembly fragment and not included in the amplification sequence. Thus the first strategy (FIG. 9A) can be more cost efficient and less error prone for synthesizing shorter starting nucleic acids (e.g., on a chip). The first strategy also uses shorter universal primers (for amplifying the fragments) and thus further reduces costs. After restriction enzyme digestion, the end pieces to be removed from the central assembly fragments are also shorter and thus are easier, cheaper and faster to remove in the first strategy than the second.

Enzymatic digestions of DNA with type IIS or other site-specific restriction enzymes typically generate an overhang of four to six nucleotides. It is unexpectedly shown in this invention, that these short cohesive ends are sufficient for ligating multiple nucleic acid fragments containing complementary termini to form the target nucleic acid. Conventionally to ensure efficiency, a ligation reaction typically involves two fragments as ligation efficiency significantly decreases with three or more fragments. In addition, longer cohesive ends are required by conventional methods to improve specificity as mismatch often occurs. Furthermore, to select for the correct ligation product, a labor-intensive and time-consuming cloning and screening process is required.

The present invention provides for, among other things: (1) successful ligation of multiple fragments (e.g., at least 4, at least 5, at least 6, at least 7, at least 8, or more) in a single reaction (e.g. single pool); (2) quick and inexpensive ligation reaction (e.g., 30 minutes at room temperature); (3) high specificity which discriminates mismatches; and (4) quick PCR step to select the correct product, without requiring cloning and screening. Another advantage of the present invention is the ability to directly use synthetic oligonucleotides of commercially available chips or microarray to construct any target nucleic acid of interest, which can be of any sequence and/or any length (e.g., at least 500 bp, at least 1 kb, at least 2 kb, at least 5 kb, at least 10 kb, or longer). Such synthetic oligonucleotides can be of substantially the same size (e.g., about 50 bases, about 100 bases, about 200 bases, about 300 bases, or longer), and thus afford ease to handle.

In one example, assuming each oligonucleotide or fragment on the chip has a payload of 100 nucleotides and the fragments have 4-base overhangs, if the number of fragments is n, then ligation product length=(n*100)-(4*(n-1)), with (n-1) ligation junctions. It should be noted that to ensure ligation specificity, the overhangs can be selected or designed to be unique for each ligation site; that is, each pair of complementary overhangs for two fragments designed to be adjacent in an assembled product should be unique and differ from any other pair of complementary overhangs by at least one nucleotide.

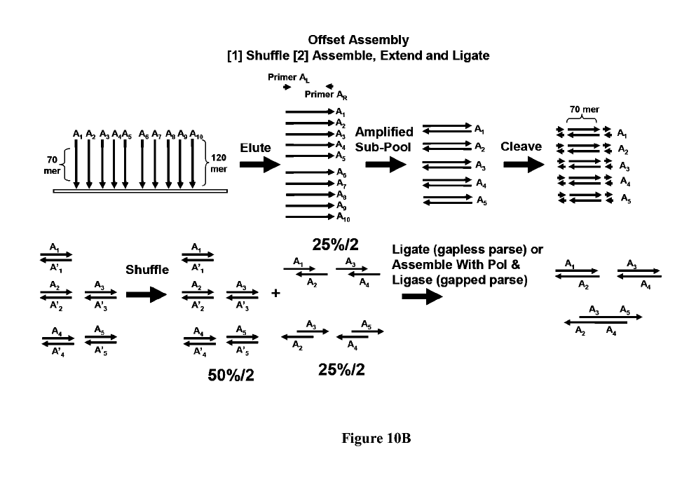

Another strategy (offset assembly) for exposing cohesive ends is illustrated in FIG. 10A. Starting from a chip, a plurality of oligos (e.g., A₁-A₁₀) can be synthesized. The oligos can be designed to have central assembly sequences which when assembled properly, form the target nucleic acid 5′-A₁-A₃-A₅-A₇-A₉-3′ (reverse strand being 3′-A₂-A₄-A₆-A₈-A₁₀-5′). That is, two adjacent oligonucleotides Aₙ and Aₙ₋₁ can be designed to overlap. As used herein, adjacent oligonucleotides refers to oligonucleotides wherein a first oligonucleotide is at the 5′ end or 3′ end of a second oligonucleotide along the linear nucleic acid sequence. In some embodiments, adjacent oligonucleotides can be contiguous. As used herein, contiguous oligonucleotides refers to two oligonucleotides wherein the first oligonucleotide ends at position arbitarily set at −1 and the second fragment starts at position arbitarily set at 0 along the linear nucleic acid sequence. The central assembly sequences can be of any desirable length such as about 50-500 nucleotides, about 60-300 nucleotides, about 70-200 nucleotides, or shorter or longer. The plurality of oligos can have uniform length for ease of handling. By way of example, the synthesized oligos can also include amplification sequences at either end, which can have restriction sites built in. The amplification sequences can be about 10-30 nucleotides, about 15-25 nucleotides, or shorter or longer. FIG. 10A shows 70-mer central assembly sequences and 120-mer overall oligos. Synthesized oligos can be eluted, cleaved, or otherwise released from the chip, and subjected to PCR amplification using primer pair A_{L} and A_{R}. Amplified products can be cleaved (e.g., with a restriction enzyme) to remove the amplification sequences (arrow heads), and the central 70-mer double-stranded assembly sequences can be purified therefrom. These double-stranded assembly sequences can then be melted (e.g., at 95° C.) and re-annealed (e.g., at 65° C.) in a single shuffling step. After shuffling of the single-stranded oligonucleotides, 25% of the products will be offset assembly products (e.g., A₁/A₂, A₂/A₃, A₃/A₄, A₄/A₅, etc.) having cohesive ends. These cohesive ends can be assembled together (stepwise or in a single reaction hierarchically) using a ligase, thereby forming the target nucleic acid 5′-A₁-A₃-A₅-A₇-A₉-3′ (reverse strand being 3′-A₂-A₄-A₆-A₈-A₁₀-5′). It should be appreciated that the oligos can also be designed such that the target nucleic acid is 5′-A₁ . . . A₃ . . . A₅ . . . A₇ . . . A₉-3′ (i.e., gaps are allowed between Aₙ and Aₙ₊₂, which can be filled using Aₙ₊₁ sequence as template). To that end, a polymerase and dNTPs can be used to extend and fill the gaps before ligation.

A second offset assembly strategy is illustrated in FIG. 10B, where a single combined assembly-(extension)-ligation step may be used, as opposed to two separate steps (i.e. assembly step and ligation step). For example, after the shuffling step (e.g., melting at 95° C. and re-annealing at 65° C.), gapless parse oligonucleotides can be ligated to form a full length product or a subassembly-product. If gaps are present in the parse, oligonucleotides can be incubated in presence of a polymerase and dNTPs to fill the gaps by chain extension prior to ligation. In some embodiments, the gapped parse can be subjected simultaneously to polymerase chain extension and ligation. As used herein the term “subassembly” refers to a nucleic acid molecule that has been assembled from a set of construction oligonucleotides. Preferably, a subassembly is at least about 2-fold, 3-fold, 4-fold, 5-fold, 10-fold, 20-fold, 50-fold, 100-fold, or more, longer than the construction oligonucleotides.

Other methods for generating cohesive ends can also be used. For example, a polymerase based method (e.g., T4 DNA polymerase) can be used to synthesize desirable cohesive ends. Regardless of the method of generating specific overhangs (e.g., complementary overhangs for nucleic acids designed to be adjacent in an assembled nucleic acid product), overhangs of different lengths may be designed and/or produced. In some embodiments, long single-stranded overhangs (3′ or 5′) may be used to promote specificity and/or efficient assembly. For example, a 3′ or 5′ single-stranded overhang may be longer than 8 bases long, e.g., 8-14, 14-20, 20-25, 25-50, 50-100, 100-500, or more bases long.

### High Fidelity Assembly

According to aspects of the invention, a plurality of nucleic acid fragments may be assembled in a single procedure wherein the plurality of fragments is mixed together under conditions that promote covalent assembly of the fragments to generate a specific longer nucleic acid. According to aspects of the invention, a plurality of nucleic acid fragments may be covalently assembled in vitro using a ligase. In some embodiments, 5 or more (e.g., 10 or more, 15 or more, 15 to 20, 20 to 25, 25 to 30, 30 to 35, 35 to 40, 40 to 45, 45 to 50, 50 or more, etc.) different nucleic acid fragments may be assembled. However, it should be appreciated that any number of nucleic acids (e.g., 2, 3, 4, 5, 6, 7, 8, 9, 10, 11, 12, 13, 14, 15, 16, 17, 18, 19, 20, etc.) may be assembled using suitable assembly techniques. Each nucleic acid fragment being assembled may be between about 100 nucleotides long and about 1,000 nucleotides long (e.g., about 200, about 300, about 400, about 500, about 600, about 700, about 800, about 900). However, longer (e.g., about 2,500 or more nucleotides long, about 5,000 or more nucleotides long, about 7,500 or more nucleotides long, about 10,000 or more nucleotides long, etc.) or shorter nucleic acid fragments may be assembled using an assembly technique (e.g., shotgun assembly into a plasmid vector). It should be appreciated that the size of each nucleic acid fragment may be independent of the size of other nucleic acid fragments added to an assembly. However, in some embodiments, each nucleic acid fragment may be approximately the same size or length (e.g., between about 100 nucleotides long and about 400 nucleotides long). For example, the length of the oligonucleotides may have a median length of between about 100 nucleotides long and about 400 nucleotides long and vary from about, +/−1 nucleotides, +/−4 nucleotides, +/−10 nucleotides. It should be appreciated that the length of a double-stranded nucleic acid fragment may be indicated by the number of base pairs. As used herein, a nucleic acid fragment referred to as “x” nucleotides long corresponds to “x” base pairs in length when used in the context of a double-stranded nucleic acid fragment. In some embodiments, one or more nucleic acids being assembled in one reaction (e.g., 1-5, 5-10, 10-15, 15-20, etc.) may be codon-optimized and/or non-naturally occurring. In some embodiments, all of the nucleic acids being assembled in one reaction are codon-optimized and/or non-naturally occurring.

In some aspects of the invention, nucleic acid fragments being assembled are designed to have overlapping complementary sequences. In some embodiments, the nucleic acid fragments are double-stranded nucleic acid fragments with 3′ and/or 5′ single-stranded overhangs. These overhangs may be cohesive ends that can anneal to complementary cohesive ends on different nucleic acid fragments. According to aspects of the invention, the presence of complementary sequences (and particularly complementary cohesive ends) on two nucleic acid fragments promotes their covalent assembly. In some embodiments, a plurality of nucleic acid fragments with different overlapping complementary single-stranded cohesive ends are assembled and their order in the assembled nucleic acid product is determined by the identity of the cohesive ends on each fragment. For example, the nucleic acid fragments may be designed so that a first nucleic acid has a first cohesive end that is complementary to a first cohesive end of a second nucleic acid and a second cohesive end that is complementary to a first cohesive end of a third nucleic acid. A second cohesive end of the second nucleic acid may be complementary to a first cohesive end of a fourth nucleic acid. A second cohesive end of the third nucleic acid may be complementary a first cohesive end of a fifth nucleic acid. And so on through to the final nucleic acid. According to aspects of the invention, this technique may be used to generate a linear arrangement containing nucleic acid fragments assembled in a predetermined linear order (e.g., first, second, third, forth, . . . , final).

In certain embodiments, the overlapping complementary regions between adjacent nucleic acid fragments are designed (or selected) to be sufficiently different to promote (e.g., thermodynamically favor) assembly of a unique alignment of nucleic acid fragments (e.g., a selected or designed alignment of fragments). Surprisingly, under proper ligation conditions, difference by as little as one nucleotide affords sufficient discrimination power between perfect match (100% complementary cohesive ends) and mismatch (less than 100% complementary cohesive ends). As such, 4-base overhangs can allow up to (4̂4+1)=257 different fragments to be ligated with high specificity and fidelity.

It should be appreciated that overlapping regions of different lengths may be used. In some embodiments, longer cohesive ends may be used when higher numbers of nucleic acid fragments are being assembled. Longer cohesive ends may provide more flexibility to design or select sufficiently distinct sequences to discriminate between correct cohesive end annealing (e.g., involving cohesive ends designed to anneal to each other) and incorrect cohesive end annealing (e.g., between non-complementary cohesive ends).

To achieve such high fidelity assembly, one or more suitable ligases may be used. A ligase may be obtained from recombinant or natural sources. In some embodiments, T3 DNA ligase, T4 DNA ligase, T7 DNA ligase, and/or E. coli DNA Ligase may be used. These ligases may be used at relatively low temperature (e.g., room temperature) and particularly useful for relatively short overhangs (e.g., about 3, about 4, about 5, or about 6 base overhangs). In certain ligation reactions (e.g., 30 min incubation at room temperature), T7 DNA ligase can be more efficient for multi-way ligation than the other ligases. A heat-stable ligase may also be used, such as one or more of Tth DNA ligase; Pfu DNA ligase; Taq ligase, any other suitable heat-stable ligase, or any combination thereof.

In some embodiments, two or more pairs of complementary cohesive ends between different nucleic acid fragments may be designed or selected to have identical or similar sequences in order to promote the assembly of products containing a relatively random arrangement (and/or number) of the fragments that have similar or identical cohesive ends. This may be useful to generate libraries of nucleic acid products with different sequence arrangements and/or different copy numbers of certain internal sequence regions.

One should appreciate that the variation in the concentration of individual fragments to be assembled might result into the assembly of incomplete intermediate constructs. For example, in the assembly of the target nucleic acid sequence (ABCDEF) using oligonucleotides A, B, C, D, E, F, each of which having the appropriate cohesive overhang end, if the concentration of the individual fragments is not equimolar (e.g. if the concentration of A, B and C is greater than the concentration of D, E and F), terminating species (such as AB and BC) can be formed resulting in a mixture of unligated intermediate products. To avoid the formation of incomplete intermediate constructs, the target nucleic acid can be assembled from at least two pools of individual fragments (e.g. pool 1: A, C, E and Pool 2: B, D, F). In some embodiments, each of the two pools comprises a plurality of nucleic acid fragments, each nucleic acid fragment of the first pool having a terminal end complementary to a terminal end of a nucleic acid fragment in the second pool. In some embodiments, the at least two pools can be formed by splitting the population of oligonucleotides into the at least two pools and amplifying the oligonucleotides in each pool separately. In other embodiments, the at least two pools can be formed by releasing (e.g. by eluting, cleaving or amplifying) oligonucleotides from a first oligonucleotide array into a first pool and releasing the oligonucleotides of a second oligonucleotide array into a second pool. Yet in an other embodiment, the at least two different pools can be formed by amplifying oligonucleotide sequences using at least two different sets of amplification tags as described herein. By the way of example, the second pool comprising oligonucleotides B, D and F can be diluted such as the molar concentration of the oligonucleotides B, D, and F present in the second pool is lower than the molar concentration of oligonucleotides A, C, and E present in the first pool. For example, the molar concentration of the oligonucleotides in the second pool may be about two times, 10 times, 20 times, 50 times, 100 times or more lower than the molar concentration of the oligonucleotides in the first pool. After mixing and ligating the two pools, the resulting product comprises the target nucleic acid having the predetermined sequence and can be separated from the excess oligonucleotides form the first pool. In certain embodiments, it may be desirable to form pools of oligonucleotide dimers having different molar concentrations. For example, the assembly of the target nucleic acid sequences ABCDEFGH can be carried out using at least two different pools, the first pool comprising oligonucleotides A, B, E. F and the second pool comprising oligonucleotides C, D, G, H. The second pool can be diluted such that the molar concentration of oligonucleotides C, D, G, H is lower (e.g. 10 times or 100 times) than the molar concentration of oligonucleotides A, B, E, F. Oligonucleotides having the appropriate cohesive overhang ends can be ligated to form the intermediate products AB and EF in the first pool and CD and GH in the second pool. Since the molar concentration of C, D, G, H is lower than the molar concentration of A, B, E. F, the molar concentration of CD and GH is lower than the molar concentration of AB and EF. After mixing the intermediates products AB, CD, EF, GH under ligating conditions, the resulting product comprising the target nucleic acid having the predetermined sequence can be separated from the excess dimers AB and EF.

In some embodiments, the nucleic acid fragments are mixed and incubated with a ligase. It should be appreciated that incubation under conditions that promote specific annealing of the cohesive ends may increase the frequency of assembly (e.g., correct assembly). In some embodiments, the different cohesive ends are designed to have similar melting temperatures (e.g., within about 5° C. of each other) so that correct annealing of all of the fragments is promoted under the same conditions. Correct annealing may be promoted at a different temperature depending on the length of the cohesive ends that are used. In some embodiments, cohesive ends of between about 4 and about 30 nucleotides in length (e.g., cohesive ends of about 5, about 10, about 15, about 20, about 25, or about 30 nucleotides in length) may be used. Incubation temperatures may range from about 20° C. to about 50° C. (including, e.g., room temperature). However, higher or lower temperatures may be used. The length of the incubation may be optimized based on the length of the overhangs, the complexity of the overhangs, and the number of different nucleic acids (and therefore the number of different overhangs) that are mixed together. The incubation time also may depend on the annealing temperature and the presence or absence of other agents in the mixture. For example, a nucleic acid binding protein and/or a recombinase may be added (e.g., RecA, for example a heat stable RecA protein).

The resulting complex of nucleic acids may be subjected to a polymerase chain reaction, in the presence of a pair of target-sequence specific primers, to amplify and select for the correct ligation product (i.e., the target nucleic acid). Alternatively, the resulting complex of nucleic acids can be ligated into a suitable vector and transformed into a host cell for further colony screening.

### Sequence Analysis and Fragment Design and Selection

Aspects of the invention may include analyzing the sequence of a target nucleic acid and designing an assembly strategy based on the identification of regions, within the target nucleic acid sequence, that can be used to generate appropriate cohesive ends (e.g., single-stranded overhangs). These regions may be used to define the ends of nucleic acid fragments that can be assembled (e.g., in one reaction) to generate the target nucleic acid. The nucleic acid fragments can then be provided or made (e.g., in a multiplex assembly reaction). The nucleic acid fragments can be selected such that they have a relative uniform size for ease to handle (e.g., purification).

According to some embodiments, the nucleic acid sequence can be designed and/or analyzed in a computer-assisted manner to generate a set of parsed double-stranded or single-stranded oligonucleotides. As used herein, the term “parsed” means that a sequence of target nucleic acid has been delineated, for example in a computer-assisted manner, such as to identify a series of adjacent oligonucleotide sequences. Adjacent oligonucleotides or nucleic acid fragments preferably overlap by an appropriate number of nucleotides to facilitate assembly according the methods of the invention. The oligonucleotide sequences can be individually synthesized and assembled using the methods of the invention.

In some embodiments, a target nucleic acid sequence may be analyzed to identify regions that contain at least one different nucleotide on one strand of the target nucleic acid. These regions may be used to generate cohesive ends. It should be appreciated that the length of a cohesive end is preferably sufficient to provide specificity. For example, cohesive ends may be long enough to have sufficiently different sequences (e.g., at least 1-base differences) to prevent or reduce mispairing between similar cohesive ends. However, their length is preferably not long enough to stabilize mispairs between similar cohesive sequences. In some embodiments, a length of about 3 to about 10 bases may be used. However, any suitable length may be selected for a region that is to be used to generate a cohesive overhang. The importance of specificity may depend on the number of different fragments that are being assembled simultaneously. Also, the appropriate length required to avoid stabilizing mispaired regions may depend on the conditions used for annealing different cohesive ends.

In some embodiments, alternating regions may be selected if they are separated by distances that define fragments with suitable lengths for the assembly design. In some embodiments, the alternating regions may be separated by about 100 to about 500 bases. However, any suitable shorter or longer distance may be selected. For example, the cohesive regions may be separated by about 200 to about 1,000 bases. It should be appreciated that different patterns of alternating regions may be available depending on several factors (e.g., depending on the sequence of the target nucleic acid, the chosen length of the cohesive ends, and the desired fragment length). In some embodiments, if several options are available, the regions may be selected to maximize the sequence differences between different cohesive ends.

Selection of the cohesive regions defines the fragments that will be assembled to generate the target nucleic acid. Accordingly, the fragment size may be between about 100 and about 500 base pairs long, between about 200 and about 1,000 bases long, or shorter or longer depending on the target nucleic acid. The fragments may be generated or obtained using any suitable technique. In some embodiments, each fragment may be assembled (e.g., in a multiplex duplex assembly reaction) so that it is flanked by double-stranded regions that can be used to generate the cohesive single-stranded regions.

In some embodiments, methods for enabling the assembly of a target polynucleotide based upon information of the sequence of the target nucleic acid. In some embodiments, a computer software can be used to parse the target sequence (e.g. A₁-Aₙ) breaking it down into a set of overlapping oligonucleotides (A₁, A₂, A₃, . . . Aₙ) of specified length. Oligos A₁, A₂, A₃, . . . Aₙ can be synthesized from a chip or microarray. In some embodiments, the oligonucleotide sequences can may be designed to include: amplification primer sequence, recognition site for a restriction enzyme, such as a type IIS restriction enzyme, padding, payload, padding, reverse complement of the recognition site for a restriction enzyme (same or different), reverse complement of a different amplification primer sequence. The payload can be an overlapping subset of the target gene (or any arbitrary nucleic acid sequence). The payload can be padded, if desired, with m nucleotides M (Mₘ) to allow the generation of a uniquely complementary cohesive ends after cleavage with the restriction enzyme(s). The primers allow amplification. The recognition sites for the restriction enzyme(s) allow the primers to be cleaved off from the payload.

In certain embodiments, it is advantageous to use the same recognition site across multiple target sequences. However, it should be noted that if a target sequence already contains the recognition site, then the oligo which contains that recognition site (in a left-to-right or right-to-left parse) will be cut, preventing correct assembly. In some embodiments, if the target sequence only contains a single occurrence of the recognition site, the problem can be solved by starting the parse within the site, and parsing one set of oligos to the left, and the other set to the right of the recognition site. Since the site will be split between 2 oligos, it will not exist as an intact sequence and thus will not be recognized or cut. If there is a desired oligo length or range of lengths, the last oligo in each side of the parse can be padded with an appropriate number m of nucleotides M (Mₘ).

This approach can be extended to more than one occurrence of a recognition site if those restriction sites appear within an integer multiple of the allowed length range for a payload. As an example of the simplest case (and ignoring any desired overlap for purposes of this example), if any portion of 2 restriction sites are exactly 100 by apart for a desired 100 by payload size, then parsing from within either one will automatically split the other. If the payload can vary from 90-110 bp, then a pair of restriction sites within this distance range can be accommodated. With this same payload range, a pair could also be split at longer distances: 180-220 bp, 270-330 bp, etc.

When parsing a target sequence into oligos, the length of the last oligo (or last in each direction if parsing from the interior) may fall outside the desired range of oligo lengths. The last oligo can be padded to the desired length. This may come however at the cost of producing additional base pairs that are otherwise not useful, specially when a large number of target sequences are assembled. In some embodiments, a solution to this problem is to concatenate every target sequence into a single long pseudo-target (with optional primer sequences between the actual target sequences), and then split into smaller, overlapping fragments of the desired length (e.g., by cleavage or amplification by PCR). The computation of the length of a fragment is presented below:

length=(pieces*max oligo length)−(junctions*overlap)

where junctions=pieces−1

For example:

length 484=(pieces 5*max oligo length 100)−(junctions 4*overlap 4)

length 504=(pieces 5*max oligo length 104)−(junctions 4*overlap 4)

If some of the target sequences contain a restriction site, then in some cases, the order in which the target sequences are concatenated can be chosen such as to have the restriction site at a junction (and within the desired oligo length range). In the general case, additional padding can be added just to the subset of target sequences that contain the restriction site, still yielding the full benefit of eliminating the padding on the majority of target sequences.

Examples of the present invention show that certain ligase enzymes in certain conditions correctly distinguishing 2 oligos with overhangs having the same last base and different second-to-last base. In some embodiments, it may be desirable to design the oligos such that the last base in each overhang is unique. Unique A, C, G, T at the end (4 junctions) allow ligation of up to 5 pieces, which is a commercially useful number to assemble. Larger numbers of ligation pieces are also contemplated in the present invention, as exemplified below:
last 2 bases unique: 4̂2=16 junctions, up to 17 pieces
last 3 bases unique: 4̂3=64 junctions, up to 65 pieces
last 4 bases unique: 4̂4=256 junctions, up to 257 pieces

Aspects of the invention relate to algorithms to parse the input target nucleic acid sequence. In some embodiments, algorithms can be used to ensure that the last base (or last 2, 3 or 4 bases) of the plurality of oligos is unique. For example, algorithms of the invention can be used to define a plurality of parsed oligonucleotides that together comprise the target sequence (naturally occurring, non-naturally occurring, or any arbitrary nucleic acid sequence, the oligonucleotides having approximately the same length and with a 4 base overlap the last base (or last 2, 3 or 4 bases) being unique. Yet in some embodiments, the oligonucleotides can be defined such as the second-to-last or third-to-last, etc or combinations thereof is unique.

In some embodiments, a first algorithm comprises the following design or decomposition steps:
Step 1: is to move over by the target amount, e.g. 100 bp,
Step 2: store the relevant 1-4 bases in a set (e.g., in a memory),
Step 3: back up by the overlap (4 bp),
Step 4: move again. For this second and each subsequent move by 100 bp, if the relevant 1-4 bases already exist in the set, then shift over 1 base at a time until encountering a 1-4 base sequence that is not yet in the set.
Step 5: add the new 1-4 base sequence to the set,
Step 6: then repeat. If the desired number of pieces is reached before reaching the end of the DNA sequence, then start over with a new set, backing up by an appropriate overlap for assembly of fragments (which may or may not be a different method than assembly of oligos into a fragment).

One skilled in the art will note that the 1-base shift could vary in direction, e.g., always left (shorter) if the nominal length is a maximum desired length, always right (longer) if the nominal length is a minimum desired length, or some combination thereof. To center around the nominal length, the shift could alternate, e.g., check positions in the following order: −1, +1, −2, +2, etc. The shift could also be weighted to prefer, for example, shorter but allow longer, e.g., −1, −2, +1, −3, −4, +2, etc.

This algorithm may be limited to design of certain target sequences, as the required shift may be large since the degrees of freedom are reduced with each subsequent addition to the set. For example, the first end may be an “A”, but the last end may not have an “A” either within several bases, thus making the last oligo very short or very long, which may be undesirable. One solution to this problem is to store an array of data for each junction, then choose either the fewest number of oligos to shift, or the least total shift distance among all oligos, or some combination thereof.

The statistics for how often any given short sequence (e.g. for a restriction site) will appear in a random 1,000 by sequence is as follows. For example, if a 6-bp restriction site is used which does not parse from the middle of a target sequence, then 22% of sequences could not be built with that restriction site. With the same 6-bp site and parsing from the middle, only the 3% of sequences that contain 2 sites could not be built (or would require additional parsing). More particularly:
If a single occurrence a restriction site prevented building:
   With quantity 1 of length 5 bp, 62% will have at least 1 site
   With quantity 1 of length 6 bp, 22% will have at least 1 site
   With quantity 1 of length 7 bp, 6% will have at least 1 site
If parsing from the interior allows 2 occurrences:
   With quantity 1 of length 5 bp, 25% will have at least 2 sites
   With quantity 1 of length 6 bp, 3% will have at least 2 sites
   With quantity 1 of length 7 bp, <1% will have at least 2 sites (about 0.2%)
If more than one restriction enzyme (and corresponding site) is used and if allowing a single occurrence:
   With quantity 2 of length 5 bp, 38% will have at least 1 site
   With quantity 2 of length 6 bp, 5% will have at least 1 site
   With length 7 bp and length 6 bp, 1% will have at least 1 site
   With quantity 3 of length 5 bp, 24% will have at least 1 site
   With quantity 3 of length 6 bp, 1% will have at least 1 site
If more than one restriction enzyme, allowing 2 occurances:
   With quantity 2 of length 5 bp, 6% will have at least 2 sites
   With quantity 2 of length 6 bp, <1% will have at least 2 sites (about 0.06%)
   With quantity 3 of length 5 bp, 2% will have at least 2 sites.

### Applications

Aspects of the invention may be useful for a range of applications involving the production and/or use of synthetic nucleic acids. As described herein, the invention provides methods for assembling synthetic nucleic acids with increased efficiency. The resulting assembled nucleic acids may be amplified in vitro (e.g., using PCR, LCR, or any suitable amplification technique), amplified in vivo (e.g., via cloning into a suitable vector), isolated and/or purified. An assembled nucleic acid (alone or cloned into a vector) may be transformed into a host cell (e.g., a prokaryotic, eukaryotic, insect, mammalian, or other host cell). In some embodiments, the host cell may be used to propagate the nucleic acid. In certain embodiments, the nucleic acid may be integrated into the genome of the host cell. In some embodiments, the nucleic acid may replace a corresponding nucleic acid region on the genome of the cell (e.g., via homologous recombination). Accordingly, nucleic acids may be used to produce recombinant organisms. In some embodiments, a target nucleic acid may be an entire genome or large fragments of a genome that are used to replace all or part of the genome of a host organism. Recombinant organisms also may be used for a variety of research, industrial, agricultural, and/or medical applications.

Many of the techniques described herein can be used together, applying suitable assembly techniques at one or more points to produce long nucleic acid molecules. For example, ligase-based assembly may be used to assemble oligonucleotide duplexes and nucleic acid fragments of less than 100 to more than 10,000 base pairs in length (e.g., 100 mers to 500 mers, 500 mers to 1,000 mers, 1,000 mers to 5,000 mers, 5, 000 mers to 10,000 mers, 25,000 mers, 50,000 mers, 75,000 mers, 100,000 mers, etc.). In an exemplary embodiment, methods described herein may be used during the assembly of an entire genome (or a large fragment thereof, e.g., about 10%, 20%, 30%, 40%, 50%, 60%, 70%, 80%, 90%, or more) of an organism (e.g., of a viral, bacterial, yeast, or other prokaryotic or eukaryotic organism), optionally incorporating specific modifications into the sequence at one or more desired locations.

Any of the nucleic acid products (e.g., including nucleic acids that are amplified, cloned, purified, isolated, etc.) may be packaged in any suitable format (e.g., in a stable buffer, lyophilized, etc.) for storage and/or shipping (e.g., for shipping to a distribution center or to a customer). Similarly, any of the host cells (e.g., cells transformed with a vector or having a modified genome) may be prepared in a suitable buffer for storage and or transport (e.g., for distribution to a customer). In some embodiments, cells may be frozen. However, other stable cell preparations also may be used.

Host cells may be grown and expanded in culture. Host cells may be used for expressing one or more RNAs or polypeptides of interest (e.g., therapeutic, industrial, agricultural, and/or medical proteins). The expressed polypeptides may be natural polypeptides or non-natural polypeptides. The polypeptides may be isolated or purified for subsequent use.

Accordingly, nucleic acid molecules generated using methods of the invention can be incorporated into a vector. The vector may be a cloning vector or an expression vector. In some embodiments, the vector may be a viral vector. A viral vector may comprise nucleic acid sequences capable of infecting target cells. Similarly, in some embodiments, a prokaryotic expression vector operably linked to an appropriate promoter system can be used to transform target cells. In other embodiments, a eukaryotic vector operably linked to an appropriate promoter system can be used to transfect target cells or tissues.

Transcription and/or translation of the constructs described herein may be carried out in vitro (i.e. using cell-free systems) or in vivo (i.e. expressed in cells). In some embodiments, cell lysates may be prepared. In certain embodiments, expressed RNAs or polypeptides may be isolated or purified. Nucleic acids of the invention also may be used to add detection and/or purification tags to expressed polypeptides or fragments thereof. Examples of polypeptide-based fusion/tag include, but are not limited to, hexa-histidine (His⁶) Myc and HA, and other polypeptides with utility, such as GFP₅ GST, MBP, chitin and the like. In some embodiments, polypeptides may comprise one or more unnatural amino acid residue(s).

In some embodiments, antibodies can be made against polypeptides or fragment(s) thereof encoded by one or more synthetic nucleic acids. In certain embodiments, synthetic nucleic acids may be provided as libraries for screening in research and development (e.g., to identify potential therapeutic proteins or peptides, to identify potential protein targets for drug development, etc.) In some embodiments, a synthetic nucleic acid may be used as a therapeutic (e.g., for gene therapy, or for gene regulation). For example, a synthetic nucleic acid may be administered to a patient in an amount sufficient to express a therapeutic amount of a protein. In other embodiments, a synthetic nucleic acid may be administered to a patient in an amount sufficient to regulate (e.g., down-regulate) the expression of a gene.

It should be appreciated that different acts or embodiments described herein may be performed independently and may be performed at different locations in the United States or outside the United States. For example, each of the acts of receiving an order for a target nucleic acid, analyzing a target nucleic acid sequence, designing one or more starting nucleic acids (e.g., oligonucleotides), synthesizing starting nucleic acid(s), purifying starting nucleic acid(s), assembling starting nucleic acid(s), isolating assembled nucleic acid(s), confirming the sequence of assembled nucleic acid(s), manipulating assembled nucleic acid(s) (e.g., amplifying, cloning, inserting into a host genome, etc.), and any other acts or any parts of these acts may be performed independently either at one location or at different sites within the United States or outside the United States. In some embodiments, an assembly procedure may involve a combination of acts that are performed at one site (in the United States or outside the United States) and acts that are performed at one or more remote sites (within the United States or outside the United States).

### Automated Applications

Aspects of the methods and devices provided herein may include automating one or more acts described herein. In some embodiments, one or more steps of an amplification and/or assembly reaction may be automated using one or more automated sample handling devices (e.g., one or more automated liquid or fluid handling devices). Automated devices and procedures may be used to deliver reaction reagents, including one or more of the following: starting nucleic acids, buffers, enzymes (e.g., one or more ligases and/or polymerases), nucleotides, salts, and any other suitable agents such as stabilizing agents. Automated devices and procedures also may be used to control the reaction conditions. For example, an automated thermal cycler may be used to control reaction temperatures and any temperature cycles that may be used. In some embodiments, a scanning laser may be automated to provide one or more reaction temperatures or temperature cycles suitable for incubating polynucleotides. Similarly, subsequent analysis of assembled polynucleotide products may be automated. For example, sequencing may be automated using a sequencing device and automated sequencing protocols. Additional steps (e.g., amplification, cloning, etc.) also may be automated using one or more appropriate devices and related protocols. It should be appreciated that one or more of the device or device components described herein may be combined in a system (e.g., a robotic system) or in a micro-environment (e.g., a micro-fluidic reaction chamber). Assembly reaction mixtures (e.g., liquid reaction samples) may be transferred from one component of the system to another using automated devices and procedures (e.g., robotic manipulation and/or transfer of samples and/or sample containers, including automated pipetting devices, micro-systems, etc.). The system and any components thereof may be controlled by a control system.

Accordingly, method steps and/or aspects of the devices provided herein may be automated using, for example, a computer system (e.g., a computer controlled system). A computer system on which aspects of the technology provided herein can be implemented may include a computer for any type of processing (e.g., sequence analysis and/or automated device control as described herein). However, it should be appreciated that certain processing steps may be provided by one or more of the automated devices that are part of the assembly system. In some embodiments, a computer system may include two or more computers. For example, one computer may be coupled, via a network, to a second computer. One computer may perform sequence analysis. The second computer may control one or more of the automated synthesis and assembly devices in the system. In other aspects, additional computers may be included in the network to control one or more of the analysis or processing acts. Each computer may include a memory and processor. The computers can take any form, as the aspects of the technology provided herein are not limited to being implemented on any particular computer platform. Similarly, the network can take any form, including a private network or a public network (e.g., the Internet). Display devices can be associated with one or more of the devices and computers. Alternatively, or in addition, a display device may be located at a remote site and connected for displaying the output of an analysis in accordance with the technology provided herein. Connections between the different components of the system may be via wire, optical fiber, wireless transmission, satellite transmission, any other suitable transmission, or any combination of two or more of the above.

Each of the different aspects, embodiments, or acts of the technology provided herein can be independently automated and implemented in any of numerous ways. For example, each aspect, embodiment, or act can be independently implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the technology provided herein comprises at least one computer-readable medium (e.g., a computer memory, a floppy disk, a compact disk, a tape, etc.) encoded with a computer program (i.e., a plurality of instructions), which, when executed on a processor, performs one or more of the above-discussed functions of the technology provided herein. The computer-readable medium can be transportable such that the program stored thereon can be loaded onto any computer system resource to implement one or more functions of the technology provided herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the technology provided herein.

It should be appreciated that in accordance with several embodiments of the technology provided herein wherein processes are stored in a computer readable medium, the computer implemented processes may, during the course of their execution, receive input manually (e.g., from a user).

Accordingly, overall system-level control of the assembly devices or components described herein may be performed by a system controller which may provide control signals to the associated nucleic acid synthesizers, liquid handling devices, thermal cyclers, sequencing devices, associated robotic components, as well as other suitable systems for performing the desired input/output or other control functions. Thus, the system controller along with any device controllers together form a controller that controls the operation of a nucleic acid assembly system. The controller may include a general purpose data processing system, which can be a general purpose computer, or network of general purpose computers, and other associated devices, including communications devices, modems, and/or other circuitry or components to perform the desired input/output or other functions. The controller can also be implemented, at least in part, as a single special purpose integrated circuit (e.g., ASIC) or an array of ASICs, each having a main or central processor section for overall, system-level control, and separate sections dedicated to performing various different specific computations, functions and other processes under the control of the central processor section. The controller can also be implemented using a plurality of separate dedicated programmable integrated or other electronic circuits or devices, e.g., hard wired electronic or logic circuits such as discrete element circuits or programmable logic devices. The controller can also include any other components or devices, such as user input/output devices (monitors, displays, printers, a keyboard, a user pointing device, touch screen, or other user interface, etc.), data storage devices, drive motors, linkages, valve controllers, robotic devices, vacuum and other pumps, pressure sensors, detectors, power supplies, pulse sources, communication devices or other electronic circuitry or components, and so on. The controller also may control operation of other portions of a system, such as automated client order processing, quality control, packaging, shipping, billing, etc., to perform other suitable functions known in the art but not described in detail herein.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### EXAMPLES

FIG. 1 shows the sequence of an arbitrarily chosen, double-stranded sequence of about 836 by long. 60-bp fragments were selected and labeled 1 to 28 (fragments 1-14 are on the positive strand; fragments 15-28 on the negative strand). These 60-bp fragments were ordered from IDT (Integrated DNA Technologies, Coralville, Iowa) (“IDT oligos”), with the following flanking sequences:

GTCACTACCGCTATCATGGCGGTCTC . . . . . GAGACCAGGAGACAGGACCGACCAAA
CAGTGATGGCGATAGTACCGCCAGAG . . . . . CTCTGGTCCTCTGTCCTGGCTGGTTT

Underlined is the recognition site of BsaI-HF, which produces a 4-base overhang:

5′ . . . G G T C T C (N)₁^{▾} . . . 3′
3′ . . . C C A G A G (N)₅_{_{▴}} . . . 5′

The BsaI-HF recognition sites are flanked by universal primers which are useful for amplification of these fragments.

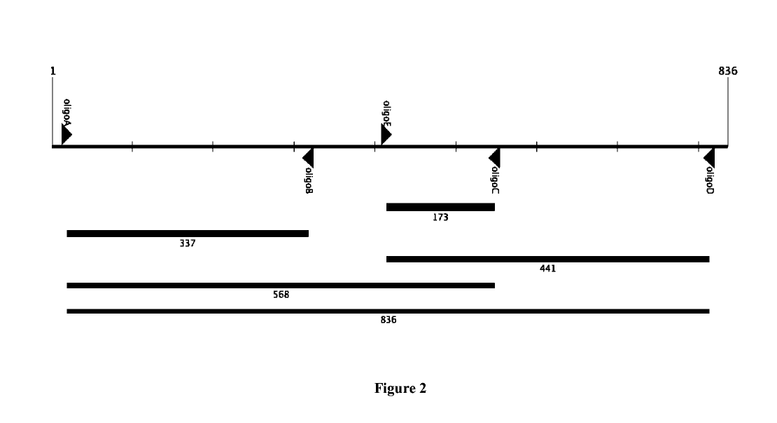

PCR primers A-E were also designed (dashed arrows in FIG. 1) for amplifying the correct ligation product. FIG. 2 shows the relative position of the primers (“oligoA” to “oligoE”) as arrowheads, as well as the predicted size of corresponding PCR products.

Double-stranded IDT oligos were subject to BsaI-HF digestion, under the following conditions:
1× NEBuffer 4
Supplemented with 100 μg/ml Bovine Serum Albumin
Incubate at 37° C.

Digested double strand oligos having cohesive ends (oligos 1-28) were purified by electrophoresis on a 4% gel. Various combinations of purified oligos 1-28 were then subject to ligation reactions. Several different ligases, temperatures and incubation times were tested for optimal ligation conditions. Ligases tested include:
T4 DNA Ligase
T4 DNA Ligase+300 mM salt (for reduced activity, higher specificity)
T3 DNA Ligase
T7 DNA Ligase
Pfu DNA Ligase
Taq DNA Ligase
E. coli DNA Ligase

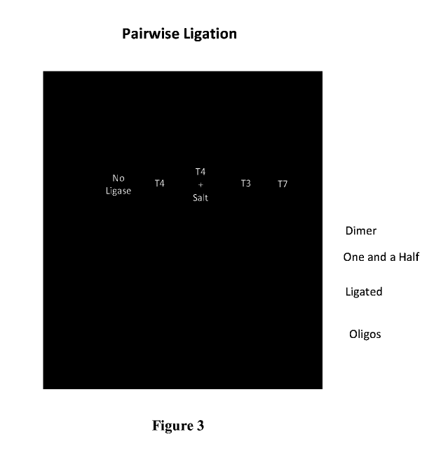

Exemplary results conducted at room temperature for 30 minutes are shown in FIGS. 3-5. FIG. 3 shows the electrophoresis results of pairwise ligation (of two oligos), from left to right of the gel: ladder, no ligase, T4 DNA ligase, T4 DNA ligase+salt, T3 DNA ligase, T7 DNA ligase. The bands from bottom to top of gel correspond to: free oligos, correct ligated product, one and a half ligated product, dimer of ligated product. T7 DNA ligase produced the most correct ligated product and thus appeared the most efficient under this experimental condition, other things being equal.

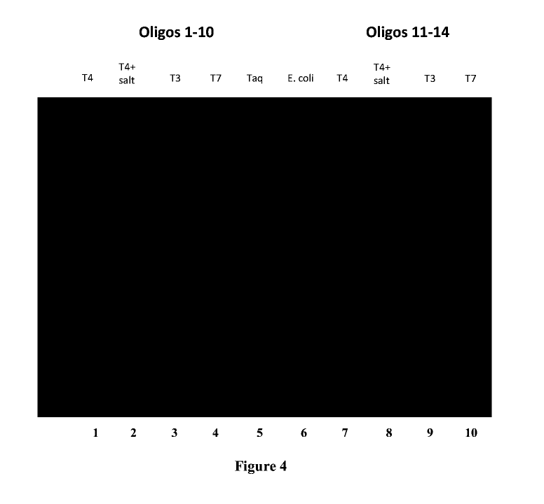

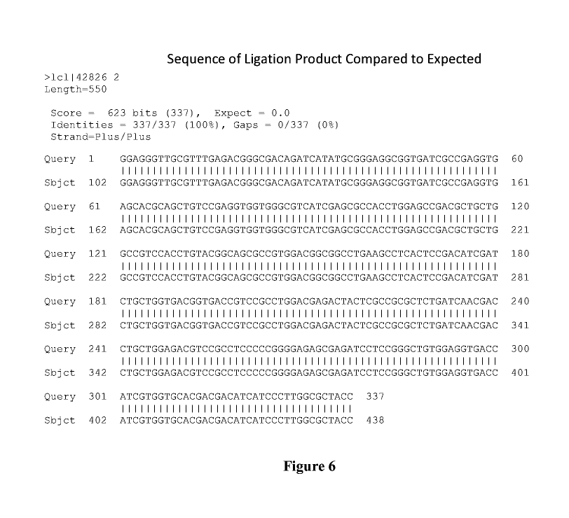

FIG. 4 shows the ligation results of oligos 1-10 (lanes 1-6) and oligos 11-14 (lanes 7-10), with different ligases indicated at the top of the gel. Multiple bands were observed, indicating the presence of different ligation products. However, upon PCR amplification using oligos A and B as primers, a strong band at about 300 by was observed. Because the predicted PCR product from oligos A and B is 337 by (see FIG. 2), this band corresponds to the correct ligation product comprising oligos 1-6 (see FIG. 1). The band was cut from the gel, purified, and sequenced. The sequencing results are shown in FIG. 6, confirming 100% fidelity of the ligation product as compared to the expected sequence. Taq DNA ligase did not produce any ligation product, probably because of the low reaction temperature (room temperature), as Taq DNA ligase is only active at elevated temperatures (45° C-65° C.).

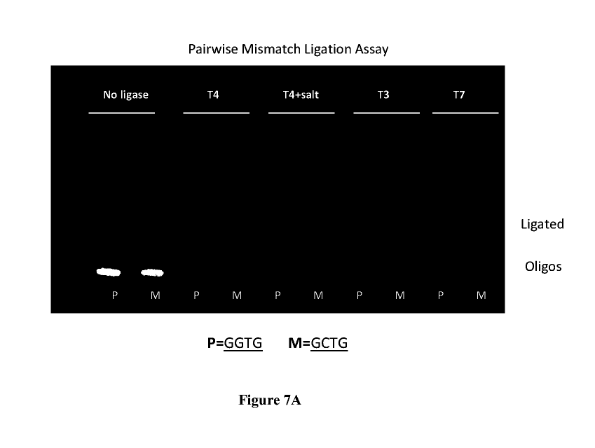

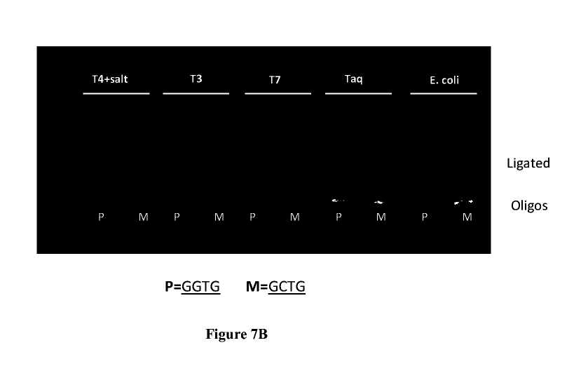

A pairwise mismatch assay was developed to test the specificity of various ligases. A pair of oligos were designed with 4-base overhangs, where the perfect match (“P”) sequence is GGTG and the mismatch (“M”) sequence is GCTG which differs from the correct sequence by one nucleotide. As shown in FIGS. 7A and 7B, two major bands can be observed, with the lower band corresponding to unligated oligos (as indicated by the no ligase controls), and the upper band corresponding to ligated product. T4 DNA ligase+salt, T3 DNA ligase, T7 DNA ligase, and E. coli DNA ligase all produced a strong band corresponding to the ligated product when using the perfect match overhangs. By contrast, when mismatch overhangs were used, majority of the product was unligated oligos. These experiment show that under these reaction conditions, T4 DNA ligase+salt, T3 DNA ligase, T7 DNA ligase, and E. coli DNA ligase all demonstrated high specificity and discrimination of mismatch as little as one nucleotide difference.

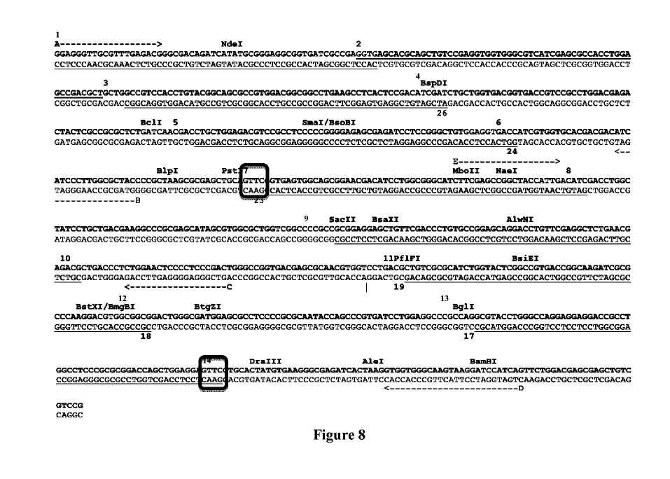

In addition to the ligation product having oligos 1-6 shown above, other ligation products were also produced, including longer products. One product appeared to have oligos 1-6 ligated to oligo 14. This is due to the fact that oligos 7 and 14 had the same cohesive end (GTTC, boxes in FIG. 8).

### EQUIVALENTS

The present invention provides among other things novel methods and devices for high-fidelity gene assembly. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### INCORPORATION BY REFERENCE

All publications, patents and sequence database entries mentioned herein are hereby incorporated by reference in their entirety as if each individual publication or patent was specifically and individually indicated to be incorporated by reference.

1. A method of producing a target nucleic acid having a predefined sequence, the method comprising:
releasing a plurality of oligonucleotides synthesized on a solid support, wherein the plurality of oligonucleotides each are engineered to comprise, at both ends, a universal primer binding site and a restriction enzyme recognition sequence;
synthesizing complementary strands of the plurality of oligonucleotides using a universal primer complementary to the universal primer binding site in a polymerase based reaction, thereby providing a plurality of blunt-end double-stranded nucleic acid fragments;
enzymatically digesting the plurality of blunt-end double-stranded nucleic acid fragments to produce a plurality of cohesive-end double-stranded nucleic acid fragments that are designed to together comprise the target nucleic acid sequence, wherein the plurality of cohesive-end double-stranded nucleic acid fragments each have two different and non-complementary overhangs; and
ligating the plurality of cohesive-end double-stranded nucleic acid fragments with a ligase, wherein a first overhang of a first cohesive-end double-stranded nucleic acid fragment is uniquely complementary to a second overhang of a second, preselected cohesive-end double-stranded nucleic acid fragment;
thereby forming a linear and predetermined arrangement of the plurality of cohesive-end double-stranded nucleic acid fragments, wherein the linear and predetermined arrangement comprises the target nucleic acid having the predefined sequence.

2. The method of claim 1, wherein the plurality of oligonucleotides are immobilized on a solid support.

3-5. (canceled)

6. The method of claim 1, wherein said the restriction enzyme recognition sequence is part of the universal primer binding site and is located at the 5′ or 3′ end of the universal primer binding site.

7. The method of claim 1, wherein the universal primer has an affinity tag to facilitate affinity removal of undesirable enzymatic digestion products.

8. The method of claim 7, wherein the affinity tag is biotin.

9. The method of claim 1, wherein the plurality of blunt-end double-stranded nucleic acids comprises at least 3, 4, 5, 6, 7, 8, 10, 15 or 20 different blunt-end double-stranded nucleic acid fragments.

10. The method of claim 1, wherein each of the plurality of blunt-end double-stranded nucleic acid fragments is at least 50, 100, 200, or 300 bases long.

11. The method of claim 1, wherein the restriction enzyme recognition sequence is the same for all blunt-end double-stranded nucleic acid fragments.

12. The method of claim 1, wherein the plurality of blunt-end double-stranded nucleic acid fragments comprise at least two different restriction enzyme recognition sequences recognizable by two different restriction enzymes that are selected to produce overhangs having the same number of bases.

13. The method of claim 1, wherein the restriction enzyme recognition sequence is capable of being recognized by a type IIs restriction enzyme.

14. The method of claim 13, wherein the type IIs restriction enzyme is BsaI, BsmBI, BspQI, BtgZI, BsmFI, FokI, BbvI, any variant thereof, or any combination thereof.

15. The method of claim 1, wherein the plurality of cohesive-end double-stranded nucleic acid fragments are designed such that a cohesive end in a cohesive-end double-stranded nucleic acid fragment is uniquely complementary to a next cohesive end in an adjacent cohesive-end double-stranded nucleic acid fragment.

16. The method of claim 1, wherein the first and second overhangs are at least 3, 4, 5, 6, 7, or 8 bases long.

17. The method of claim 1, wherein the first and second overhangs differ from one another by at least 1, 2, 3 or 4 bases.

18. The method of claim 1, wherein the first and second overhangs are 5′ or 3′ overhangs.

19. The method of claim 1, further comprising, before the ligating step, purifying the plurality of cohesive-end double-stranded nucleic acid fragments to remove undesirable enzymatic digestion products.

20. The method of claim 19, wherein the undesirable enzymatic digestion products include fragments less than about 40, about 35, about 30, about 25, about 20, or about 15 bases long.

21. The method of claim 19, wherein said purifying includes differential affinity to silica, size filtration, differential precipitation with polyethylene glycol or cetyltrimethlyammonium bromide, or any combination thereof.

22. The method of claim 1, wherein the ligase is T3 DNA ligase, T4 DNA ligase, T7 DNA ligase, E. coli DNA ligase, any variant thereof, or any combination thereof.

23. The method of claim 1, wherein the target nucleic acid is a non-naturally occurring nucleic acid.

24. The method of claim 1, wherein the target nucleic acid is at least 500, 800, 1000, 1500, 2000, or 3000 bases long.

25. The method of claim 1, further comprising amplifying the target nucleic acid using a pair of primers specific to the target nucleic acid and a polymerase.

26. The method of claim 1, further comprising confirming the sequence of the target nucleic acid.

27. The method of claim 1, wherein the plurality of blunt-end double-stranded nucleic acid fragments are hierarchically assembled from synthetic oligonucleotides.

28. The method of claim 1 wherein the plurality of cohesive-end double-stranded nucleic acid fragments are ligated in a single pool.

29. The method of claim 1 wherein the plurality of cohesive-end double-stranded nucleic acid fragments are in at least two pools, each nucleic acid fragment of the first pool having a terminal end complementary to a nucleic acid fragment of the second pool.

30-44. (canceled)
